SEQUENCE LISTING (40 sequences)

SEQ ID NO: 1 - DNA, 24 bp, Artificial Sequence
  Primer used for q-PCT testing
ctgtcttcatacgcttctgcactc

SEQ ID NO: 2 - DNA, 20 bp, Artificial Sequence
  Primer used for q-PCT testing
ccagccactaggtgccaatc

SEQ ID NO: 3 - DNA, 21 bp, Artificial Sequence
  Primer used for q-PCT testing
gggagcaaggaaagggtctta

SEQ ID NO: 4 - DNA, 21 bp, Artificial Sequence
  Primer used for q-PCT testing
cacctgcacaattctccgagt

SEQ ID NO: 5 - DNA, 22 bp, Artificial Sequence
  Primer used for q-PCT testing
ccagtcatcttgtctgcaccaa

SEQ ID NO: 6 - DNA, 24 bp, Artificial Sequence
  Primer used for q-PCT testing
gaaatgccagtgtcttcatatcca

SEQ ID NO: 7 - DNA, 21 bp, Artificial Sequence
  Primer used for q-PCT testing
cagctgctgttttgttccgaa

SEQ ID NO: 8 - DNA, 21 bp, Artificial Sequence
  Primer used for q-PCT testing
cagctcaactgaaagccgttt

SEQ ID NO: 9 - DNA, 20 bp, Artificial Sequence
  Primer used for q-PCT testing
gtctgtgatcgccggacagt

SEQ ID NO: 10 - DNA, 23 bp, Artificial Sequence
  Primer used for q-PCT testing
gtcagtcctagagcccacagaag

SEQ ID NO: 11 - DNA, 21 bp, Artificial Sequence
  Primer used for q-PCT testing
ccactctcatcaggccaagtg

SEQ ID NO: 12 - DNA, 20 bp, Artificial Sequence
  Primer used for q-PCT testing
tgaaggcagggactggagtt

SEQ ID NO: 13 - DNA, 19 bp, Artificial Sequence
  Primer used for q-PCT testing
cccgcagtgcacacctaga

SEQ ID NO: 14 - DNA, 21 bp, Artificial Sequence
  Primer used for q-PCT testing
acacagacagaagcggcaatg

SEQ ID NO: 15 - DNA, 25 bp, Artificial Sequence
  Primer used for q-PCT testing
acggcttagggtaatgtcagtacag

SEQ ID NO: 16 - DNA, 21 bp, Artificial Sequence
  Primer used for q-PCT testing
taagcaaggcaacggaatcag

SEQ ID NO: 17 - DNA, 20 bp, Artificial Sequence
  Primer used for q-PCT testing
cccaacacgcagcaaaatta

SEQ ID NO: 18 - DNA, 24 bp, Artificial Sequence
  Primer used for q-PCT testing
gcaagctgataggtcagagaacag

SEQ ID NO: 19 - DNA, 21 bp, Artificial Sequence
  Primer used for q-PCT testing
acaggaggcttcggagtttct

SEQ ID NO: 20 - DNA, 22 bp, Artificial Sequence
  Primer used for q-PCT testing
gcaatcacaccaagagggaaaa

SEQ ID NO: 21 - DNA, 21 bp, Artificial Sequence
  Primer used for q-PCT testing
aggacggtaacttggctgcat

SEQ ID NO: 22 - DNA, 21 bp, Artificial Sequence
  Primer used for q-PCT testing
gtgctgatggatgtgctggtt

SEQ ID NO: 23 - DNA, 20 bp, Artificial Sequence
  Primer used for q-PCT testing
ccccagaccaaactgctcat

SEQ ID NO: 24 - DNA, 19 bp, Artificial Sequence
  Primer used for q-PCT testing
ctcgcacttgctgctccaa

SEQ ID NO: 25 - DNA, 25 bp, Artificial Sequence
  Primer used for q-PCT testing
aacactgaagctatgcatttgaaga

SEQ ID NO: 26 - DNA, 22 bp, Artificial Sequence
  Primer used for q-PCT testing
gacttagcaacctgcagcacaa

SEQ ID NO: 27 - DNA, 24 bp, Artificial Sequence
  Primer used for q-PCT testing
tgctgtgtgtaagtggagaacttg

SEQ ID NO: 28 - DNA, 19 bp, Artificial Sequence
  Primer used for q-PCT testing
gcctttgcctcaccttgga

SEQ ID NO: 29 - DNA, 22 bp, Artificial Sequence
  Primer used for q-PCT testing
ccacatttccacaccagaactg

SEQ ID NO: 30 - DNA, 22 bp, Artificial Sequence
  Primer used for q-PCT testing
ggtgaccccttcacaagagttt

SEQ ID NO: 31 - DNA, 21 bp, Artificial Sequence
  Primer used for q-PCT testing
ggcctccatccctcttactca

SEQ ID NO: 32 - DNA, 22 bp, Artificial Sequence
  Primer used for q-PCT testing
cagctgcgtgaccttaggtgta

SEQ ID NO: 33 - DNA, 20 bp, Artificial Sequence
  Primer used for q-PCT testing
gcatgggcacctcagattgt

SEQ ID NO: 34 - DNA, 20 bp, Artificial Sequence
  Primer used for q-PCT testing
tgcccagtggacaggtttct

SEQ ID NO: 35 - DNA, 20 bp, Artificial Sequence
  Primer used for q-PCT testing
ggcaggaatgtgtggcagat

SEQ ID NO: 36 - DNA, 23 bp, Artificial Sequence
  Primer used for q-PCT testing
agcaggcactgtttacatccttt

SEQ ID NO: 37 - DNA, 23 bp, Artificial Sequence
  Primer used for q-PCT testing
gcacttggtaacaggcacatctt

SEQ ID NO: 38 - DNA, 21 bp, Artificial Sequence
  Primer used for q-PCT testing
ctctgccctcccctaaatgtc

SEQ ID NO: 39 - DNA, 25 bp, Artificial Sequence
  Primer used for q-PCT testing
caagcaaacatcctatcaacaacaa

SEQ ID NO: 40 - DNA, 21 bp, Artificial Sequence
  Primer used for q-PCT testing
ttccatcctccaccagtgttc

(19) United States
(12) Patent Application Publication
(10) Pub. No.: US 2018/0256665 A1
(43) Pub. Date: Sep. 13, 2018

(54) MULATEIRO-DERIVED COMPOSITIONS AND USE THEREOF FOR PREVENTING HAIR LOSS AND PROMOTING HAIR GROWTH

(71) Applicants: Nivaldo José MOREIRA, São Paulo (BR); Luiz Francisco PIANOWSKI, Braganca Paulista (BR); Lech W. DUDYCZ, Baltic, CT (US)

(72) Inventors: Nivaldo José MOREIRA, São Paulo (BR); Luiz Francisco PIANOWSKI, Braganca Paulista (BR); Lech W. DUDYCZ, Baltic, CT (US)

(21) Appl. No.: 15/747,968

(22) PCT Filed: Jul. 26, 2016
(86) PCT No.: PCT/US2016/044121

§ 371 Date: Jan. 26, 2018

Related U.S. Application Data
(60) Provisional application No. 62/197,535, filed on Jul. 27, 2015.

(51) Int. Cl. A61K 36/74; A61P 17/14

(52) U.S. Cl. CPC A61K 36/74; A61P 17/14

(57) ABSTRACT

Mulateiro is a plant, which is found in the Amazon, various extracts of which are used in traditional ethnic medicine. Describes are an extract of Mulateiro bark and its components, as well as their use for preventing hair loss and promoting hair growth. The major components of the extract were identified as isomers of chlorogenic acid and secoiridoids glucosides.

### FIELD

The present teachings relate to a pharmaceutical composition which can be used for preventing hair loss and promoting hair growth. The composition contains an extract of Mulateiro bark. Some of the active compounds of the extract belong to a family of secoiridoids or iridoids, and quinic acid derivatives.

### BACKGROUND

Mulateiro (Calycophyllum spruceanum) is a multi-purpose canopy tree in the Amazon. It grows tall and straight up to a height of about 30 m, and has been long used as a source of good, high density lumber. Other common names in use for the tree include ashi, asho, capirona, capirona de bajo, capirona negra, corusicao, escorregamacaco, firewood tree, mulateiro, mulateiro-da-várzea, naked tree, palo mulato, pau-marfim, pau, mulato, pau-mulato-da-várzea, uhuachaunin, haxo, huiso asho, and nahua.

Mulateiro is noted for its ability to completely shed and regenerate its bark on a yearly basis, making harvesting the bark a totally renewable and sustainable enterprise. Calycophyllum is a small genus with only about six species spread through tropical America; all are medium-sized to large trees. This particular species is indigenous to the Amazon basin in Brazil, Peru, Bolivia, and Ecuador. It is called mulateiro or pau-mulato in Brazil, and capirona in Peru.

Mulateiro is used for many purposes in traditional herbal medicine. A bark decoction is used topically for eye infections and infected wounds as well as for skin spots, skin depigmentation, wrinkles and scars. It also stops bleeding quickly and is often applied to bleeding cuts. It is also thought to soothe insect bites and reduce bruising and swelling. The bark is decocted and used internally for diabetes and disorders of the ovaries. The resin is used for abscesses, and skin tumors. Due to its beneficial effects to the skin, it is appearing as an ingredient in natural cosmetic products in Peru and Brazil.

Described below is a Mulateiro extract, including some of its active ingredients, that can be used for preventing hair loss and promoting hair growth.

### SUMMARY

The present invention via embodiments disclosed hereinafter and many other embodiments within the scope of the claims of this patent overcome the problems as set forth above and/or afford other related advantages.

In certain aspects the present disclosure describes a pharmaceutical composition for hair loss prevention or promotion of hair growth. The composition contains a Mulateiro bark extract.

In certain aspects the present disclosure describes a pharmaceutical composition for hair loss prevention or promotion of hair growth. The composition contains one or more Mulateiro bark extract components.

In certain aspects the present disclosure describes a method for preventing hair loss. The method includes administering a therapeutically effective does to a patient of a pharmaceutical composition which contains a Mulateiro bark extract.

In certain aspects the present disclosure describes a method for promoting hair growth. The includes administering a therapeutically effective dose to a patient of a pharmaceutical composition which contains a Molateiro bark extract.

### A BRIEF DESCRIPTION OF THE DRAWINGS

The present invention, including composition of matter and method aspects, is illustratively shown and described in reference to the accompanying drawings.

FIG. 1 is an illustration of the process of preparation of the Mulateiro extract of the present teachings;

FIG. 2 is a summary illustration of genes involved in hair growth control that were evaluated for attenuation by the compositions of the present teachings: genes involved in hair growth promotion are shown italics, while genes involved in hair growth inhibition are shown in a bold font;

FIG. 3 is an illustration of the effects of the compositions of the present teachings on the genes which are important during telogen to anagen phase transition;

FIG. 4 is an illustration of the effects of the compositions of the present teachings on the expression of BMP2 gene, in this and subsequent figures the effects of two different durations of treatment at two different substance concentrations are shown for each tested substance indicated along the horizontal axis: the first two columns represent the effects of treatment for 5 hours at concentrations of 50 ug/ml and 100 ug/ml, and the last two columns—for 24 hours at concentrations of 50 ug/ml and 100 ug/ml;

FIG. 5 is an illustration of the effects of the compositions of the present teachings on the expression of FGF7 gene;

FIG. 6 is an illustration of the effects of the compositions of the present teachings on the expression of PDGFA gene;

FIG. 7 is an illustration of the effects of the compositions of the present teachings on the expression of PDGFC gene;

FIG. 8 is an illustration of the effects of the compositions of the present teachings on the expression of WNT10A gene;

FIG. 9 is an illustration the effects of the compositions of the present teachings on the genes that are relevant to active anagen phase;

FIG. 10 is an illustration of the effects of the compositions of the present teachings on the expression of CTNNB1 gene;

FIG. 11 is an illustration of the effects of the compositions of the present teachings on the expression of GSK3B gene;

FIG. 12 is an illustration of the effects of the compositions of the present teachings on the expression of KRT15 gene;

FIG. 13 is an illustration of the effects of the compositions of the present teachings on the expression of LEF1 gene;

FIG. 14 is an illustration of the effects of the compositions of the present teachings on the expression of mTOR gene;

FIG. 15 is an illustration of the effects of the compositions of the present teachings on the expression of TCF3 gene;

FIG. 16 is an illustration of the effects of the compositions of the present teachings on the expression of TCF4 gene;

FIG. 17 is an illustration of the effects of the compositions of the present teachings on the expression of VDR gene;

FIG. 18 is an illustration of the effects of the compositions of the present teachings on the genes which are important during anagen to catagen phase transition;

FIG. 19 is an illustration of the effects of the compositions of the present teachings on the expression of EGF gene;

FIG. 20 is an illustration of the effects of the compositions of the present teachings on the expression of IL6 gene;

FIG. 21 is an illustration of the effects of the compositions of the present teachings on the expression of SRD5A1 gene;

FIG. 22 is an illustration of the effects of the compositions of the present teachings on the expression of SRD5A2 gene;

FIG. 23 is an illustration the effects of the compositions of the present teachings on the genes that are relevant to active catagen phase;

FIG. 24 is an illustration of the effects of the compositions of the present teachings on the expression of BAX gene;

FIG. 25 is an illustration of the effects of the compositions of the present teachings on the expression of BCL2 gene; and

FIG. 26 is an illustration of the effects of the compositions of the present teachings on the expression of H2AFX gene.

### DETAILED DESCRIPTION

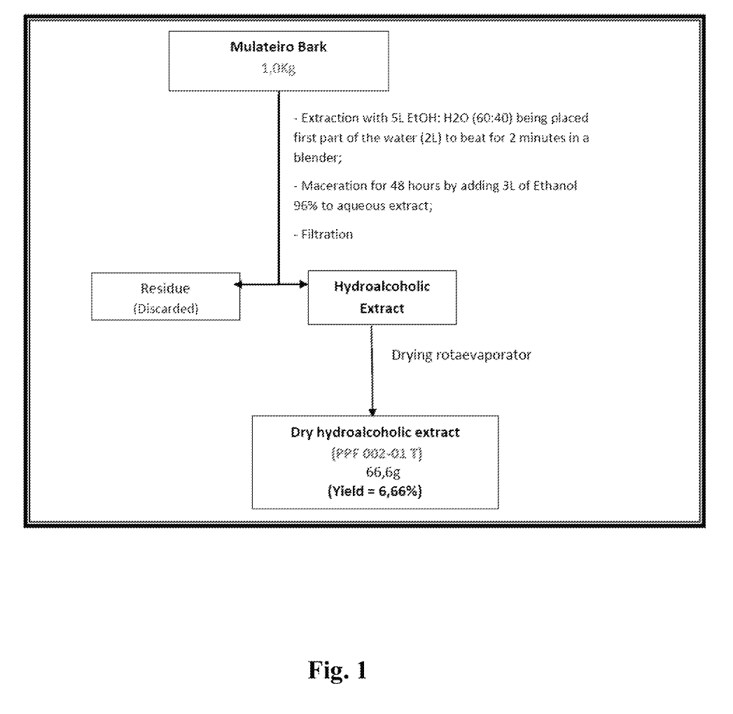

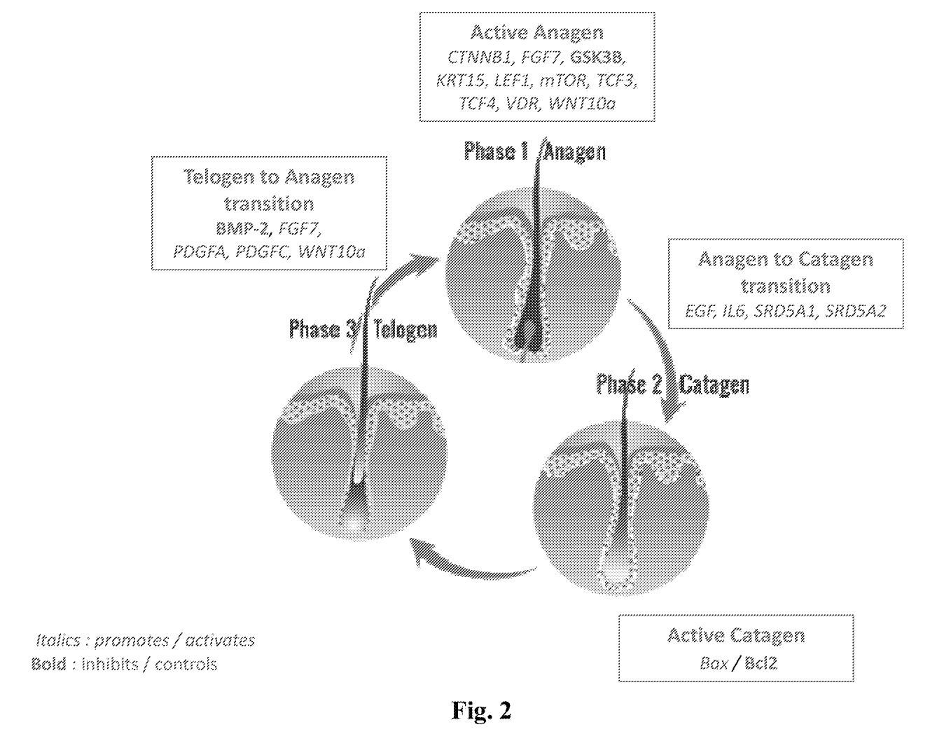

The teachings disclosed herein are based, in part, upon preparing an extract of mulateiro bark. The extract of the present teachings can be prepared, for example, according to the process illustrated in FIG. 1. The extract has been subsequently fractionated and the fractions, as well as the original extract, have been tested for their ability to attenuate gene expression of certain genes known to be relevant to hair growth. Some of the genes which are relevant to the phases of hair growth are shown in FIG. 2. With reference to FIG. 2, genes expression of which is known to promote hair growth are shown in italic font on, while genes which expression is known to inhibit hair growth are shown in bold font.

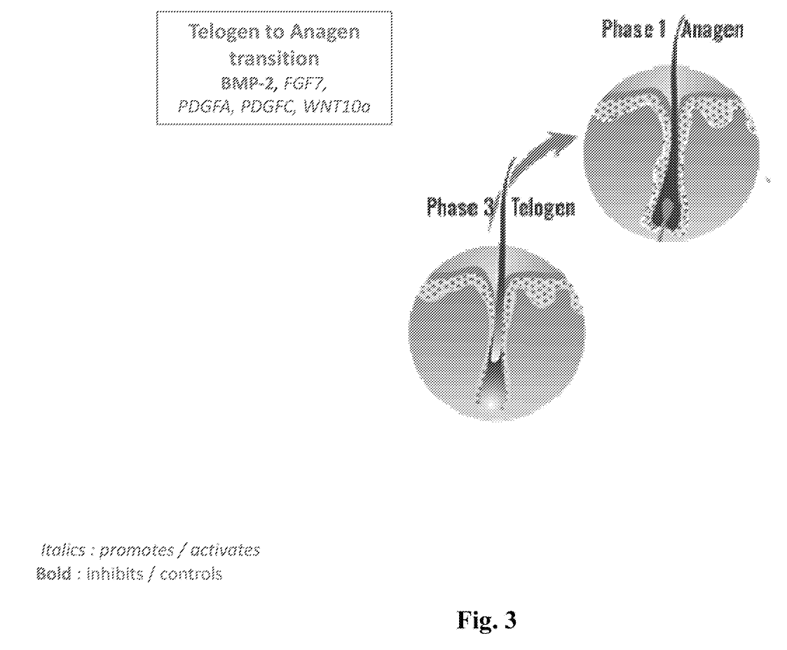

Targeted phases of the hair cycle were telogen to anagen transition, active anagen, anagen to catagen transition and finally active catagen. Genes that are relevant to telogen to anagen transition are shown in FIG. 3, and the effects of the compositions of the present teachings on these genes are summarized in Table 1.

TABLE 1
Summary of attenuating activity of the mulateiro extract of the present teachings
on the expression of genes relevant to telogen to anagen transition.

| | | |
|---|---|---|
| Gene | Activity | Role |

Telogen to Anagen transition

| | | |
|---|---|---|
| BMP-2 | − | BMP signaling maintains Hair Follicle (HF) quiescence; once |
| | | anagen is activated, its levels gradually peak at the late anagen |
| | | and telogen to inhibit proliferation. |
| FGF7 | + | Important trigger for new hair growth; gradually increases in |
| | | Dermal Papilla (DP) cells during the long 2nd telogen prior to |
| | | activation. |
| PDGFA/ | + | PDGFs trigger resting hair follicles to enter the hair growth cycle |
| PDGFC | | and induce entry into the anagen phase by activitaing DP cells to |
| | | promote hair growth. |
| WNT10a | + | WNT10a promotes anagen induction and maintenance of anagen |
| | | state. |

In Table 1 the presence of extract induced attenuating activity is indicated with a (+)-sign, the absence—with a (−)-sign.

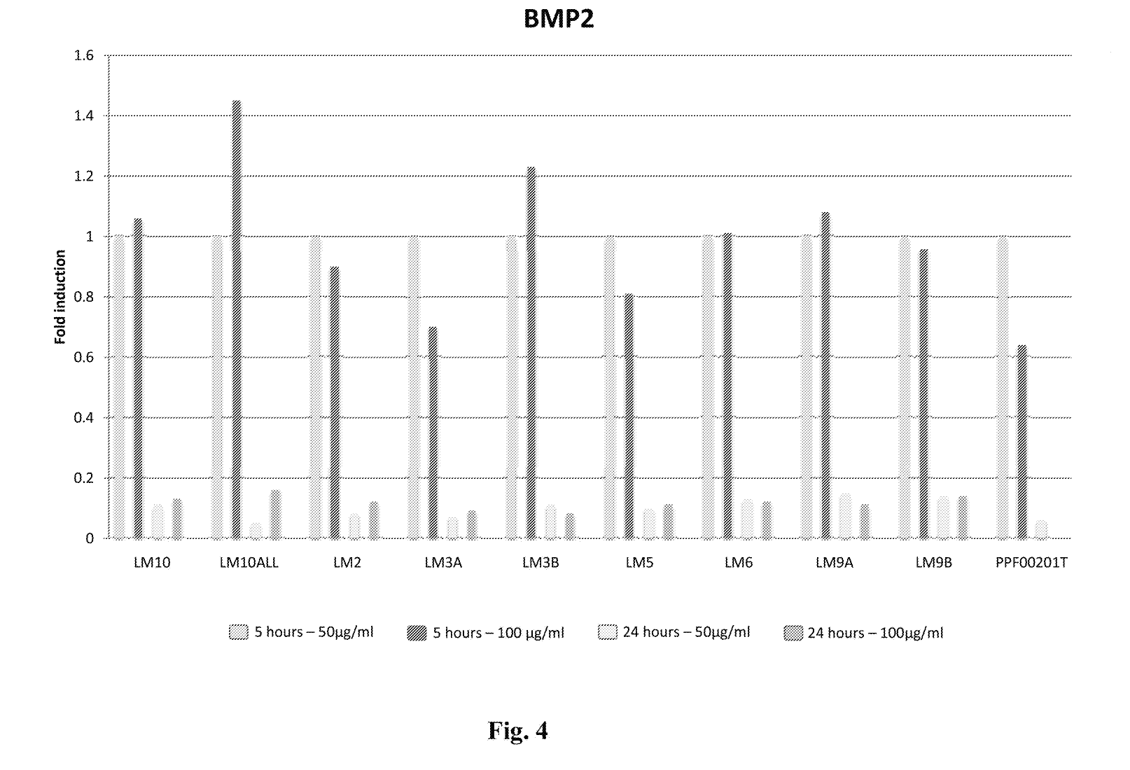

The effects of the compositions of the present teachings on BMP2 gene expression are shown in FIG. 4. BMP signaling maintains Hair Follicle quiescence. Bmp2/4 are present at low level in the mesenchyme during early anagen. These levels gradually peak at the late anagen and telogen to inhibit proliferation. BMP2 transcript is downregulated at anagen, whereas upregulated at catagen and telogen. Hair-inducing cell fate. Telogen-phase hair follicle stem cells lacking the ability to respond to BMPs immediately re-enter anagen, reinforcing the central importance of BMP signaling.

Interesting activity was observed of all tested fractions for long duration treatments (whatever the dose tested) that markedly decreased Bmp-2 expression levels (>80% inhibition);

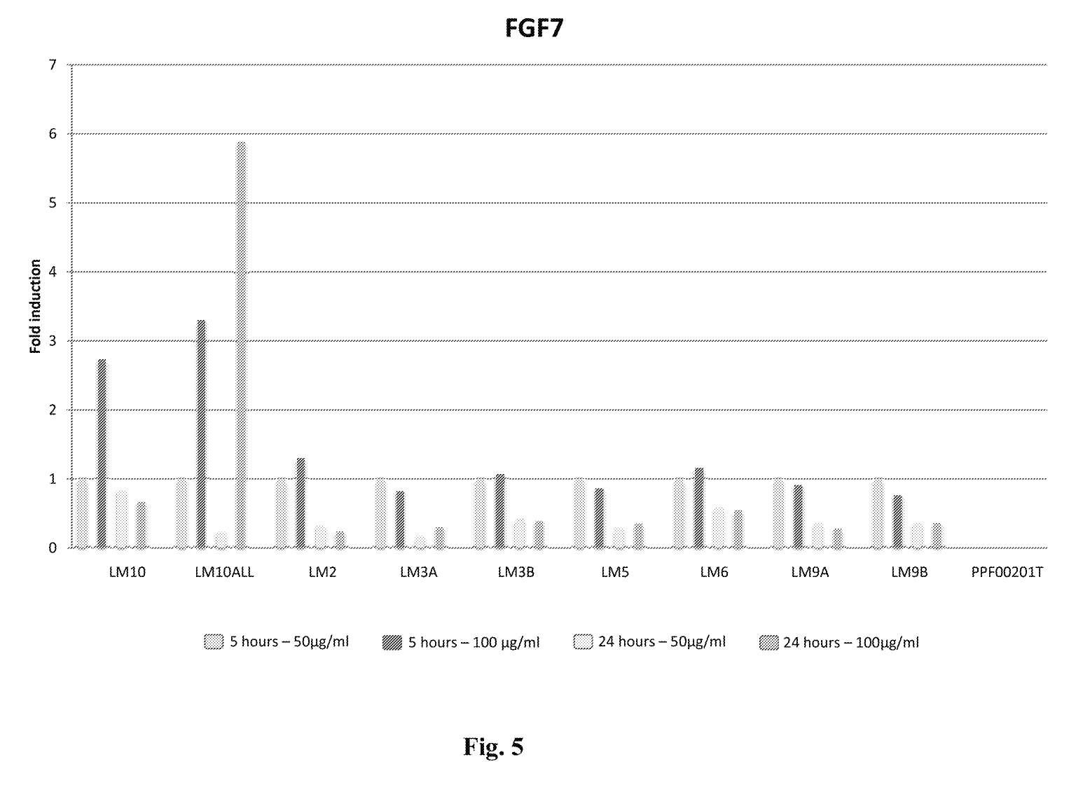

The effects of the compositions of the present teachings on FGF7 gene expression are shown in FIG. 5. Up-regulation of FGF7 is an important trigger for new hair growth. FGF7 is a keratinocyte growth factor, it prolongs the anagen phase of the hair cycle and delays progression into the catagen phase. FGF7 and FGF10 are involved in promoting hair follicle regeneration during the anagen to telogen transition. FGF7 expression gradually increases in DP during the long 2nd telogen prior to activation.

While most fractions induced a decrease in FGF7 expression, LM10ALL at 100 μg/ml and for a long duration of treatment (24 h) induces a marked increase (6 fold-induction) in FGF7 expression.

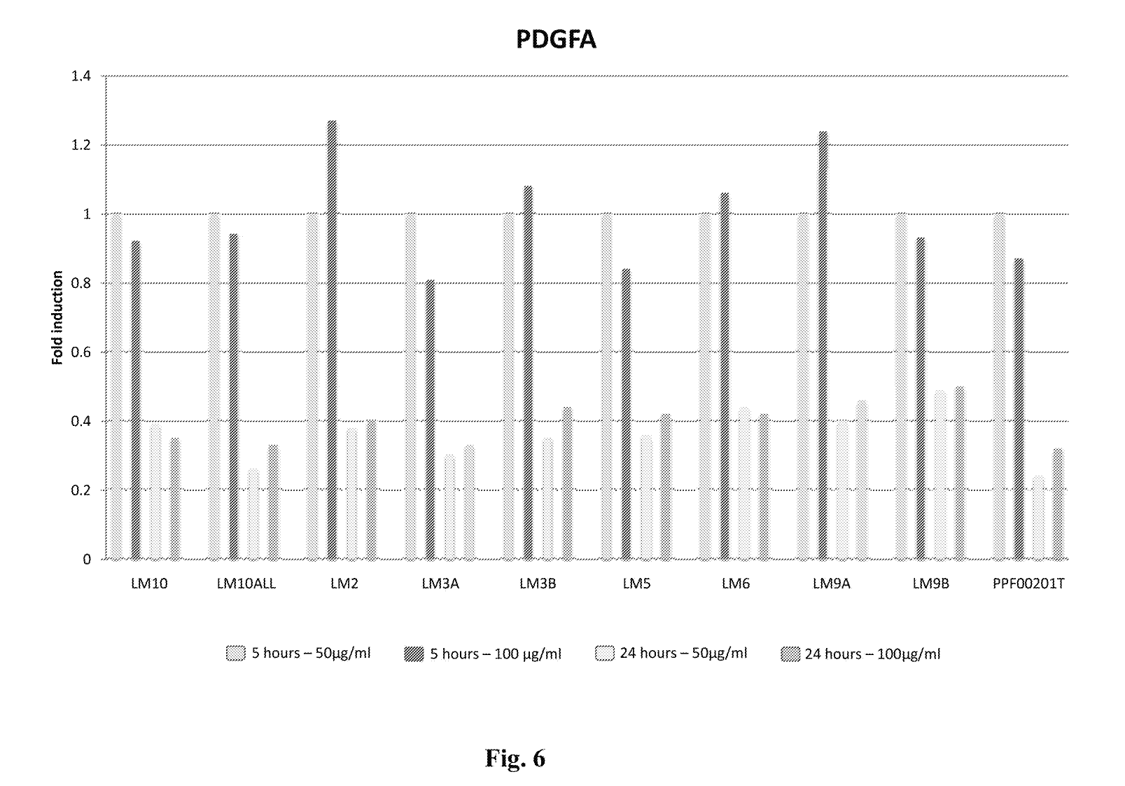

The effects of the compositions of the present teachings on PDGFA gene expression are shown in FIG. 6. PDGFA triggers resting hair follicles to enter the hair growth cycle. PDGFA may be an important factor stimulating morphogenesis of new capillaries in anagen. Platelet-derived growth factor (PDGF) has been demonstrated to induce entry into the anagen phase of the hair growth cycle. Fat-derived PDGF, in particular, was proposed to act on DP cells which in turn regulate induction of follicle regeneration in the hair cycle.

All tested fractions induced a 60% to 80% decrease in PDGFA expression for long duration treatments at both tested doses, only short duration treatments at 100 μg/ml with fractions LM2 and LM9A induces a moderate increase in PDGFA expression.

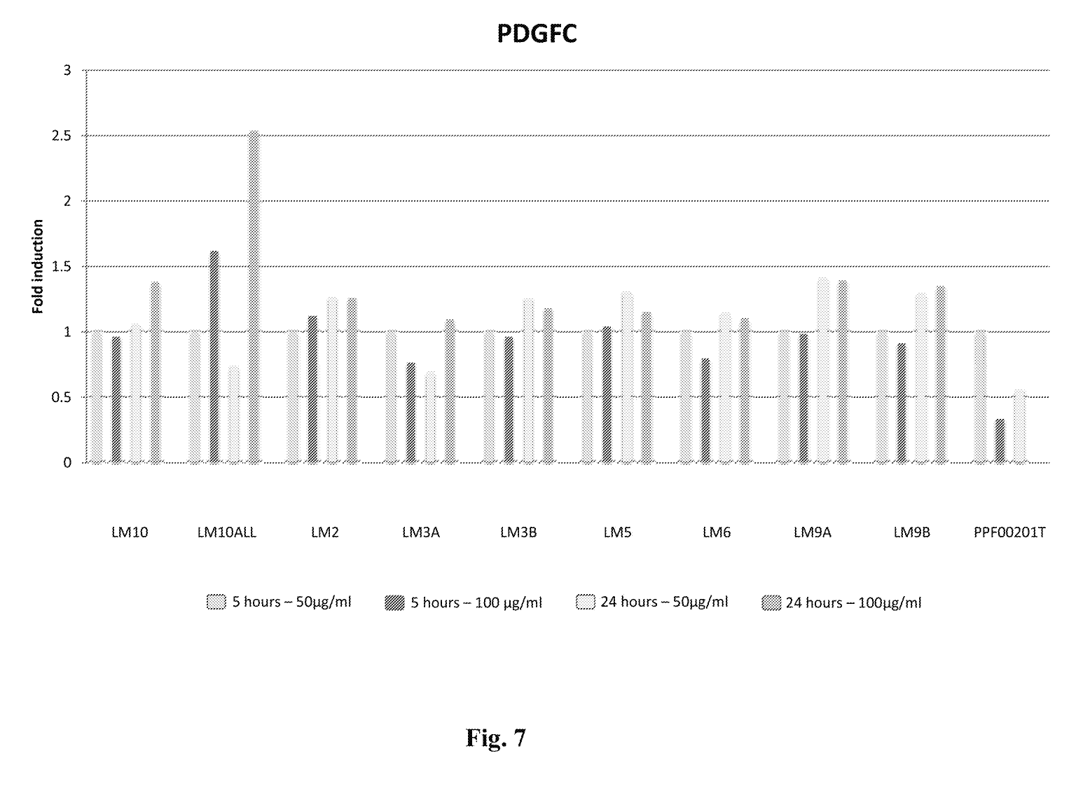

The effects of the compositions of the present teachings on PDGFC gene expression are shown in FIG. 7. PDGF signals are involved in both the epidermis-follicle interaction and the dermal mesenchyme-follicle interaction required for hair canal formation and the growth of the dermal mesenchyme, respectively.

For most tested fractions, while short duration treatment at 100 μg/ml induced little variation in PDGFC expression, long duration treatment, especially at 100 μg/ml, induced a moderate increase in PDGFC expression. Notably, LM10ALL induced a marked increase (2.5 fold induction) at the highest tested dose for long duration treatment. PPF 002-01 T induces the highest decrease observed (nearly 50%).

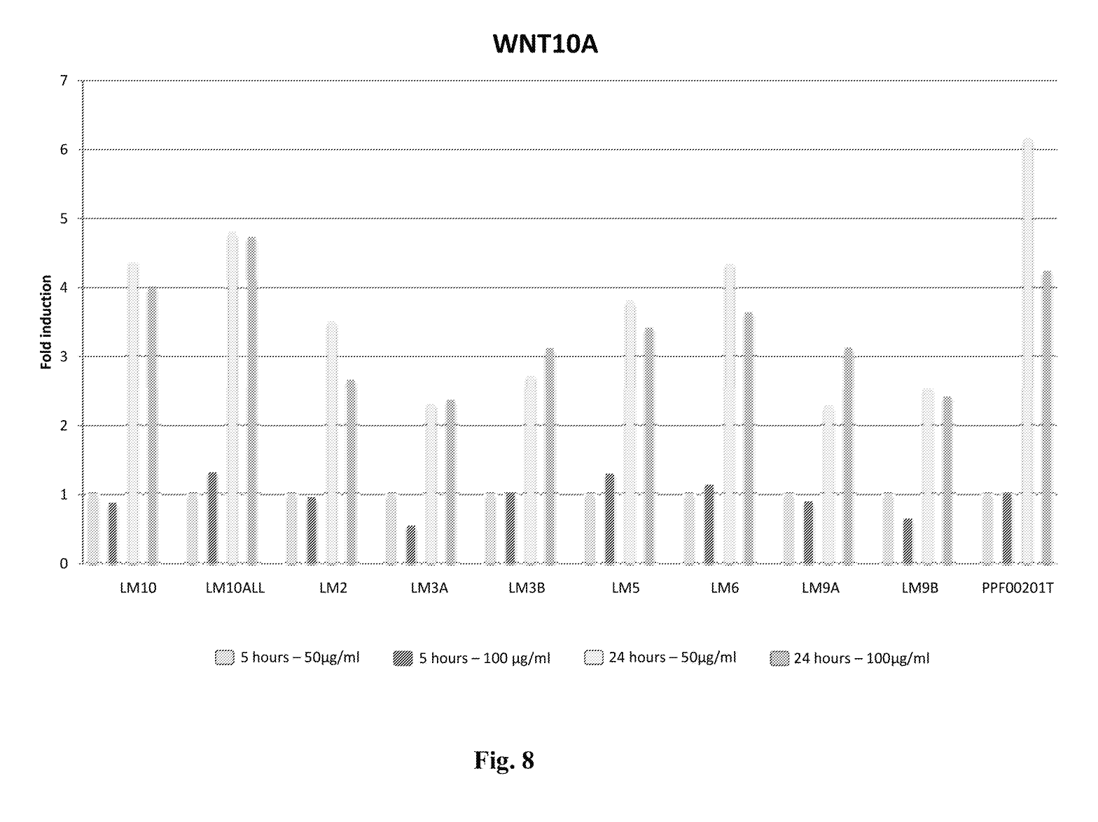

The effects of the compositions of the present teachings on WNT10a gene expression are shown in FIG. 8. In adult epidermis, the Wnt pathway controls both stem cell renewal and lineage selection of stem cells (hair follicle, sebaceous gland, interfollicular epidermis). WNT10a promotes anagen induction and maintenance of anagen state. In androgenic alopecia, the decrease in Wnt10a is linked to a delay in telogen-anagen transition and a shortening of anagen duration. Wnt transcriptional targets include the Wnt molecules themselves, Eda, Fgfs, Keratins, Lef1, Movo1, Foxn1, Msx2, Follistatin, Tgfβ2, Cyclin D1 and other cell cycle related genes

Interestingly, all tested fractions induce little change in Wnt10a expression for short duration treatment, whereas long term treatment systematically induce a significant increase. This effect is particularly marked for fractions LM10, LM10ALL, LM6 and PPF00201T that exhibit a fold induction >4.

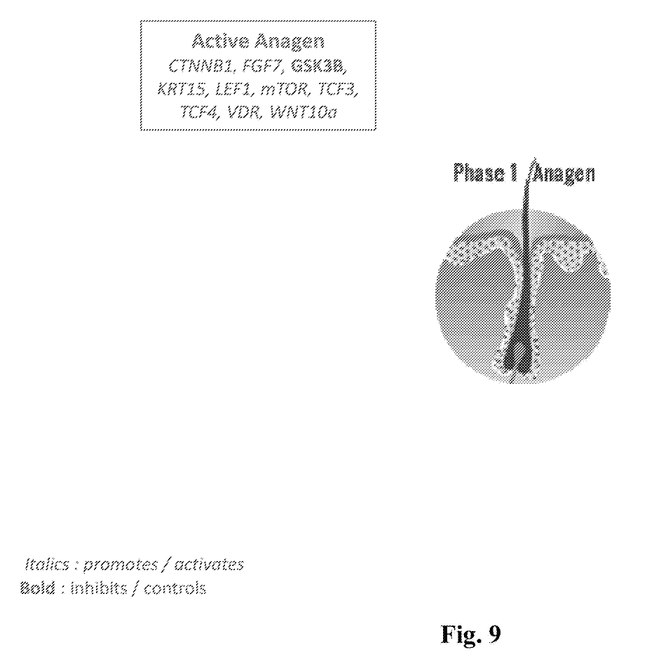

Regarding the potential of the different tested fractions to induce hair growth, genes related to active anagen phase are of utmost interest. Genes that are relevant to active anagen phase are shown in FIG. 9. The genes relevant to active anagen, expression of which was evaluated for susceptibility to mulateiro extract of the present teachings, are listed in Table 2 below. In Table 2 the presence of extract induced attenuating activity is indicated with a (+)-sign, the absence—with a (−)-sign.

TABLE 2
Summary of attenuating activity of the mulateiro extract of the present
teachings on the expression of genes relevant to active anagen.

| | | |
|---|---|---|
| Gene | Activity | Role |

Active Anagen

| | | |
|---|---|---|
| CTNNB1 | + | Downstream effector of the Wnt pathway; induces HF growth. |
| | | Stabilized β-catenin acts as a transcriptional cofactor for TCF-3, |
| | | TCF-4 and LEF1. Weakly expressed during catagen and telogen |
| FGF7 | + | FGF7 prolongs the anagen phase of the hair cycle and delays |
| | | progression into the catagen phase. |
| GSK3B | − | Its inhibition through Wnt signaling allows for the stabilization of β- |
| | | catenin and increases mTOR function. |
| KRT15 | + | Marker of anagen DP cells and of hair follicle stem cells. |
| LEF1 | + | Important signaling molecule in the Wnt pathway; binds to β- |
| | | catenin and acts as a transcription factor for downstream genes. |
| | | High levels in the hair follicle during anagen but low during |
| | | catagen and telogen. |
| mTOR | + | Plays a key role as part of the growth-promoting pathway by |
| | | which Wnt promotes the proliferation of cells and regenerative |
| | | capacity of tissue-specific stem cells. |
| TCF-3 | + | Through Wnt signalling, TCF-3 directs stem cells along the hair |
| | | lineage; in the absence of Wnt signlas, TCF-3 may maintain the |
| | | skin stem cells in an undifferentiated state. |
| TCF-4 | + | Promotes DP cells proliferation and cytokine secretory activity. |
| | | Increased TCF-4 expression promotes hair growth. |
| VDR | + | VDR is a TCF/Lef-independent transcriptionnal effector of the Wnt |
| | | pathway; it is required for β-catenin induced hair follicle |
| | | formation in adult epidermis |
| WNT10a | + | Wnt10a promotes anagen induction and maintenance of anagen |
| | | state. In androgenic alopecia, the decrease in Wnt10a is linked to |
| | | a delay in telogen-anagen transition and a shortening of anagen |
| | | duration. Its transcriptional targets include Fgfs, Keratins, Lef1, |
| | | and other cell cycle related genes. |

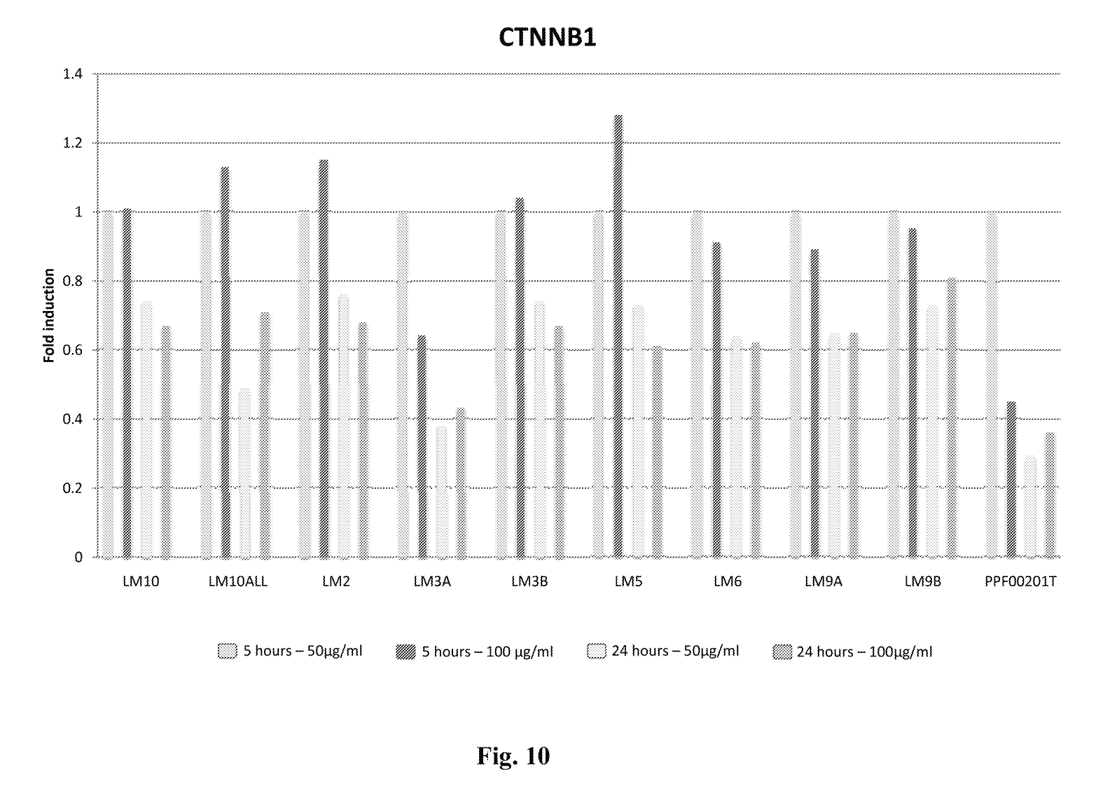

The effects of the compositions of the present teachings on CTNNB1 gene expression are shown in FIG. 10. β catenin is a downstream effector of the Wnt pathway; activation of β catenin induces growth (anagen) of existing hair follicles and induces ectopic follicles that arise from pre-existing follicles. Stabilized β-catenin acts as a transcriptional cofactor for TCF-3, TCF-4 and LEF1. β catenin is also an indispensable factor for the hair development and cycle maintenance; it is highly expressed during anagen, and weakly expressed during catagen and telogen.

Only LM10ALL, LM2 and LM5 induced a slight increase in β catenin expression when tested at the highest dose for a short duration of exposure (5 h). Long duration treatment at both tested doses systematically induces a slight to moderate decrease in β-catenin expression.

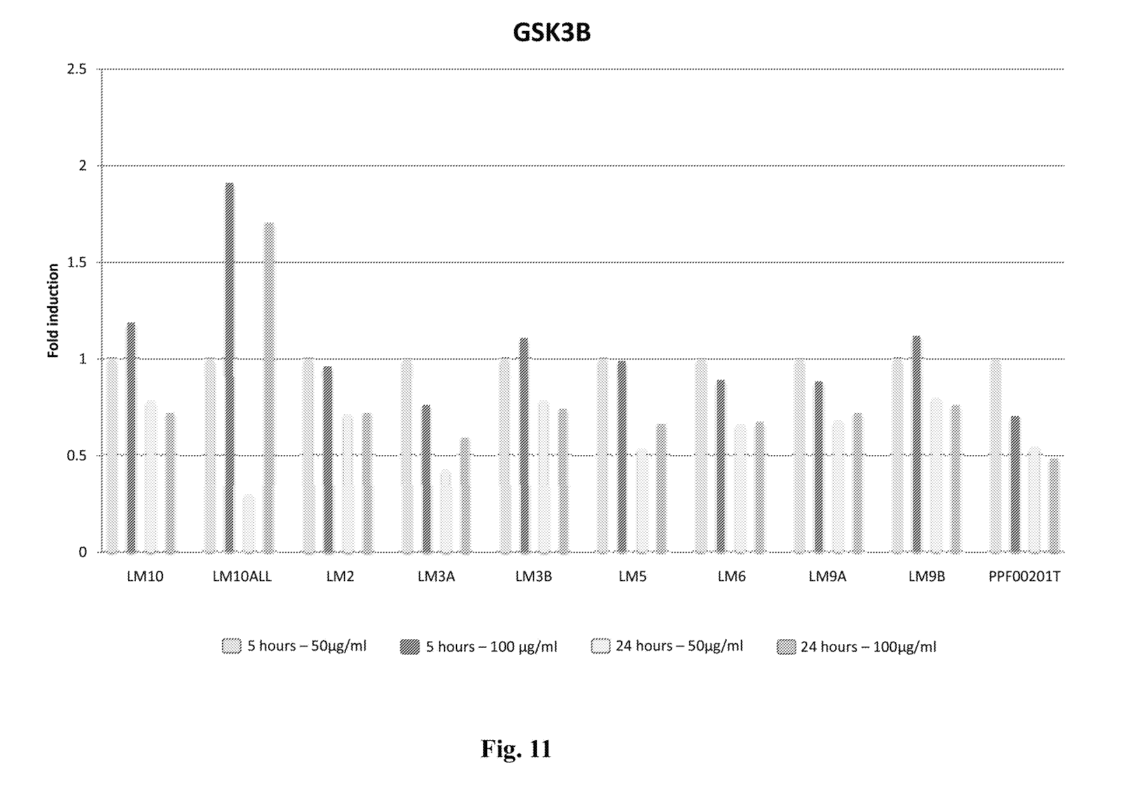

The effects of the compositions of the present teachings on GSK3B gene expression are shown in FIG. 11. Slight decrease in GSK3B expression for most tested fractions, especially for long term treatment, consistent with the increased observed in Wnt expression. Only fraction LM10ALL seems to increase GSK3 expression for the highest tested independently of the treatment duration, but results for low dose—short duration treatment not consistent with other results.

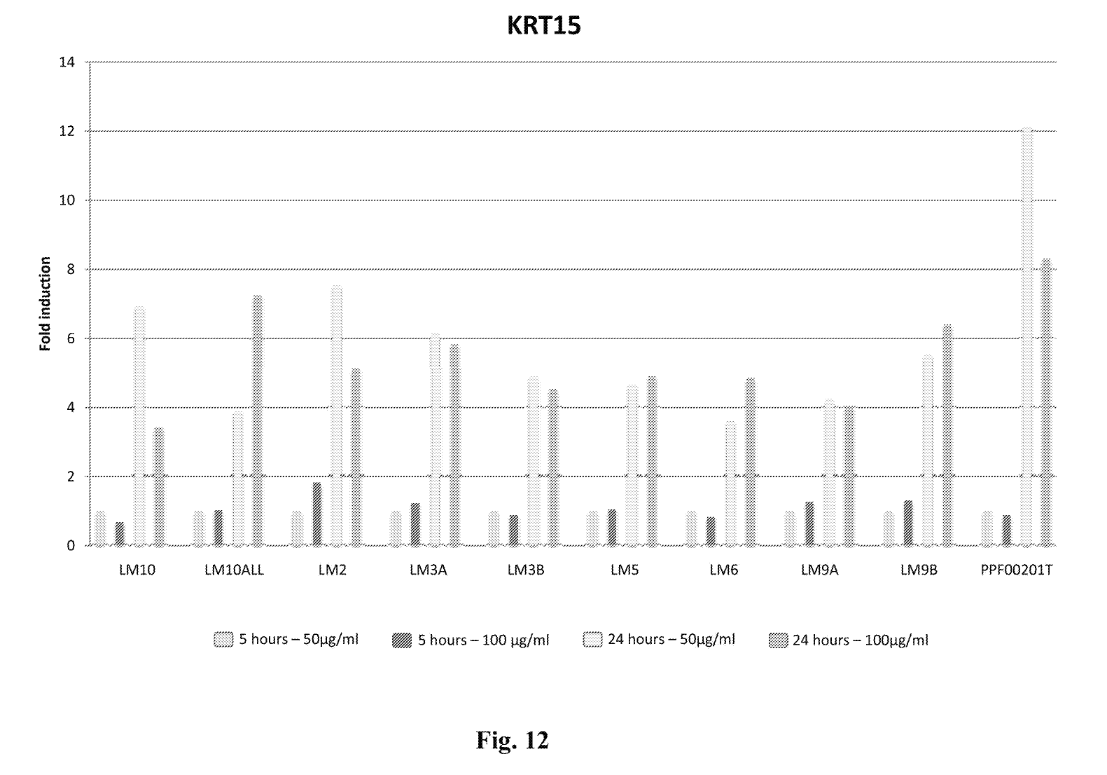

The effects of the compositions of the present teachings on KRT15 gene expression are shown in FIG. 12. KRT15 (keratin15) is a marker of anagen DP cells—the marker of hair follicle stem cells.

Keratin 15 gene expression is dramatically increased following long term treatment with all tested fractions. Lowest doses seem to be more efficient at stimulating KRT 15 expression for fractions LM10, LM2 and PPF00201T, whereas a dose dependency only appears for fraction LM10-ALL. For all other tested fractions, no marked difference is observed between doses.

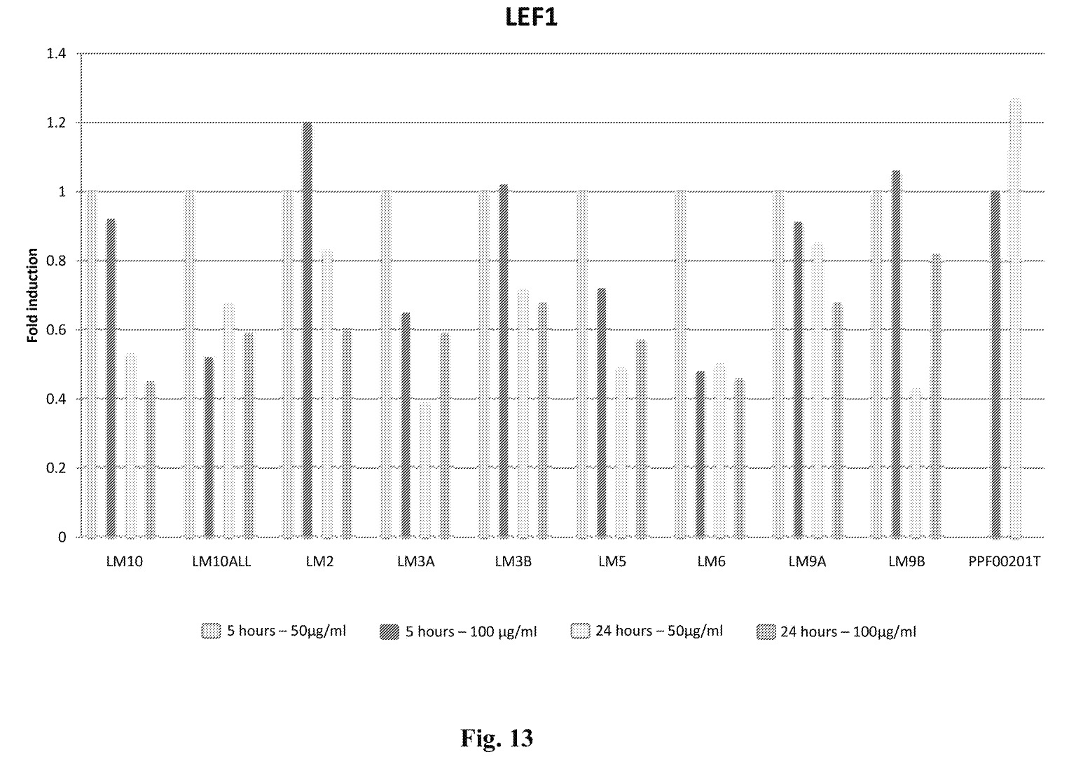

The effects of the compositions of the present teachings on LEF1 gene expression are shown in FIG. 13. Lef-1 is another important signaling molecule in the Wnt pathway. In response to a canonic Wnt signal, β-catenin accumulates in the nucleus and binds Lef-1 or other Lef/Tcf family transription factor of downstream genes. Lef-1 is found at high levels in the hair follicle during anagen, but at low levels during catagen and telogen. β-catenin protein shows a similar dynamic pattern through the hair cycle. β-catenin and Lef-1 are both necessary for the development and differentiation of hair follicles. Both proteins may have a function in maintaining the hair cycle. In addition, they appear in the same areas at the same times and show a similar pattern of changes in proteins levels. β-catenin and Lef-1 function through the formation of a β-catenin/Lef-1 complex during the cyclical growth of hair follicles.

Except for fractions LM2 (100 μg/ml—5 h) and PPF 002-01 T (50 μg/ml—24 h), all other tested fractions at both doses and both duration exposures induced a decrease in LEF1 expression. This decrease is particularly significant for fractions LM10ALL, LM3A and LM6.

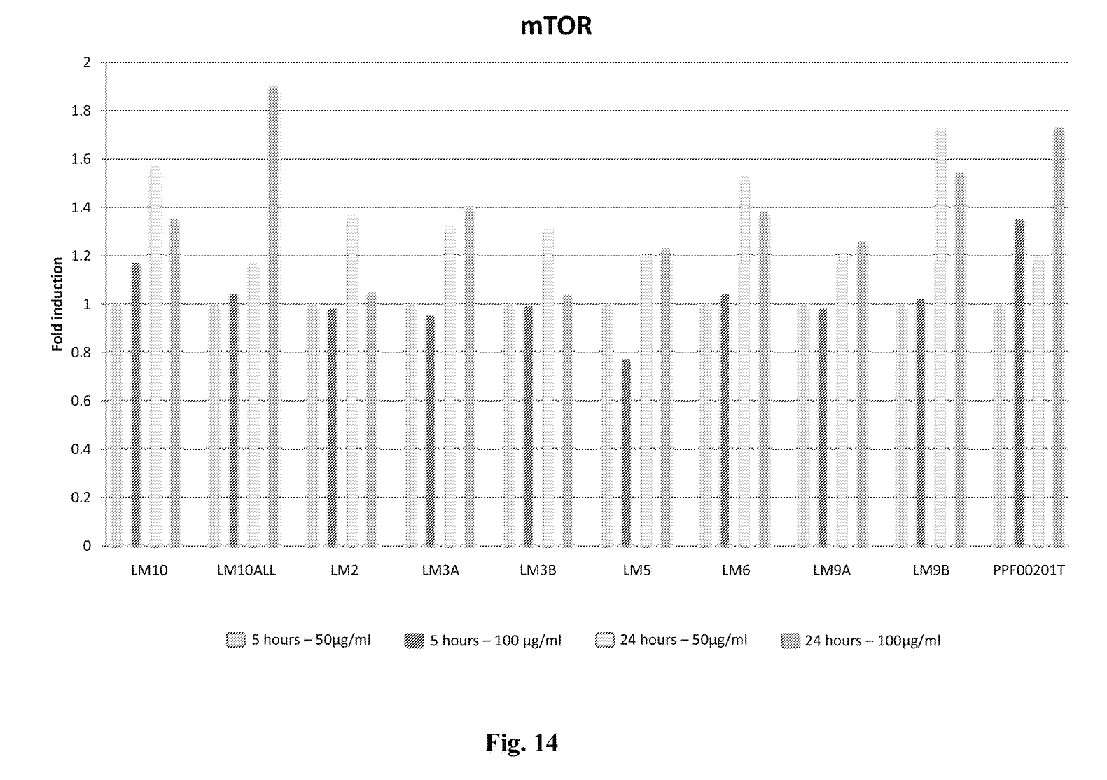

The effects of the compositions of the present teachings on mTOR gene expression are shown in FIG. 14. mTOR is a key downstream component of the pathway by which Wnt(1) activation can lead to cell growth and tissue aging. Increase in Wnt leads to inhibition of GSK3, which leads to inhibition of TSC2 (Tumor suppressor Protein 2 that exerts an inhibitory effect on mTOR), which leads to increase in mTOR function, which leads to persistent proliferation of epithelial cells, which leads to exhaustion of the HF stem cell compartment. Wnt(1) may promote the proliferation of cells and regenerative capacity of tissue-specific stem cells and mTOR may play a key role as part of the growth-promoting pathway by which Wnt acts.

No marked increase in mTOR expression was observed for short term treatments, except for fraction PPF00201T. More marked effects were observed for all fractions tested for long term treatments, and especially for LM10, LM10ALL, LM9B and PPF00201T. The results consistent with the systematic increase observed in Wnt expression, especially for long term treatments.

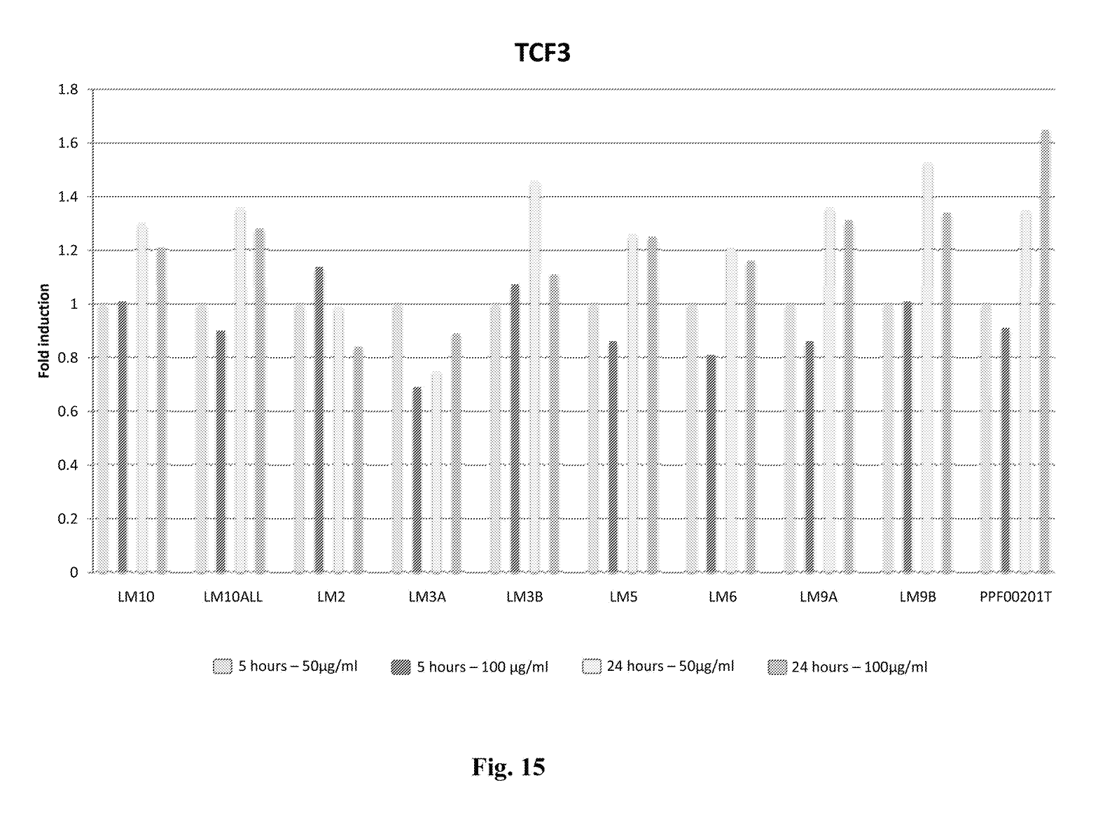

The effects of the compositions of the present teachings on TCF3 gene expression are shown in FIG. 15. TCF-3 and TCF-4 as transcriptional repressors play a crucial role in hair follicle stem cell maintenance. In the absence of Wnt signals, TCF functions in the skin stem cells to maintain an undifferentiated state. Through Wnt signaling, TCF-3 directs stem cell along the hair lineage. Therefore inhibition of Wnt signaling by Tcf3 within the stem cells and by secreted Wnt inhibitors from the stem cells and the niche appear to be crucial for maintaining stem cell quiescence, while activation of Wnt signaling is required for the transition to a new hair growth phase.

While short duration treatments at both tested induced little or no variation in TCF3 expression, long duration treatments (24 h) with all tested fractions (except LM2 and LM3A) stimulated TCF3 expression. For those fractions, no significant concentration related increase in TCF3 expression is observed.

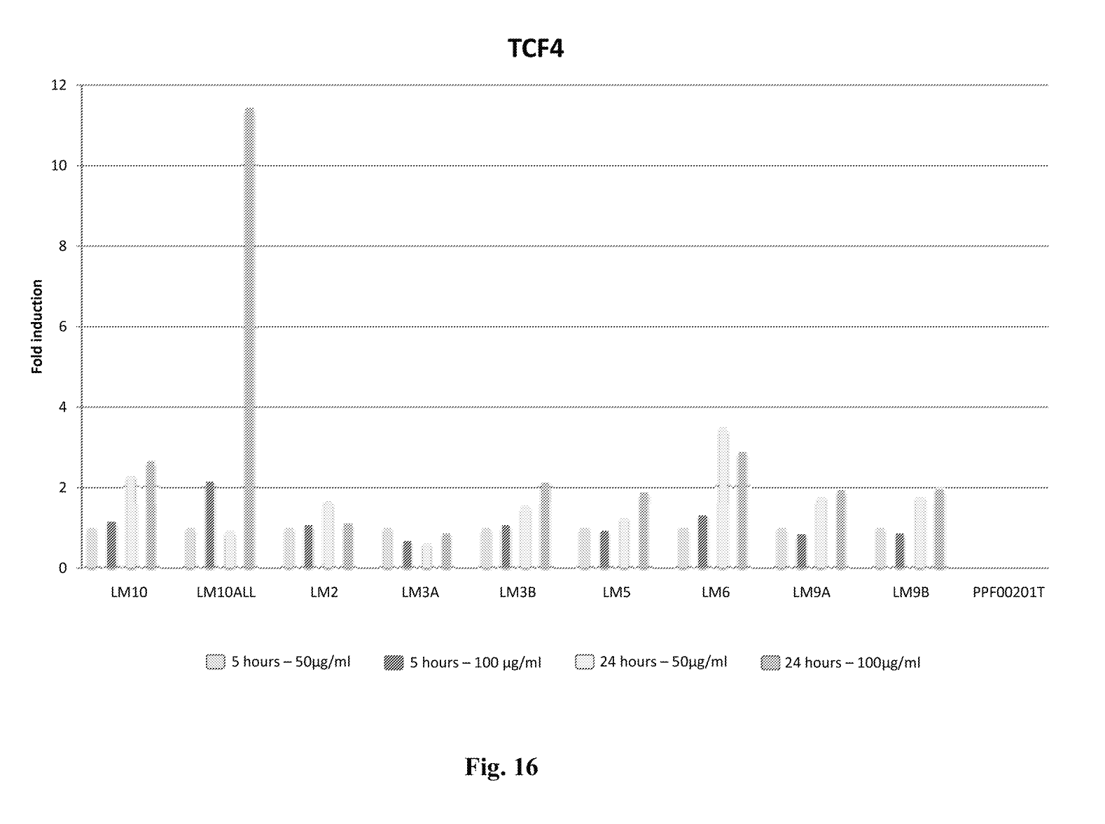

The effects of the compositions of the present teachings on TCF4 gene expression are shown in FIG. 16. TCF-4 promotes DPC (dermal papilla cells) proliferation and cytokine secretory activity. Increased TCF-4 expression promotes hair growth.

While short duration treatment at both tested doses inducef little or no variation in TCF3 expression, most tested fractions induced a significant increase in TCF4 expression for a long duration treatment (24 h). This increase is particularly marked for fractions LM10, LM6 and LM10ALL. LM10ALL induced a dramatic increase (>11 fold-induction) in TCF4 expression when tested at 100 μm/ml. Only LM3A induces a slight decrease in TCF4 expression.

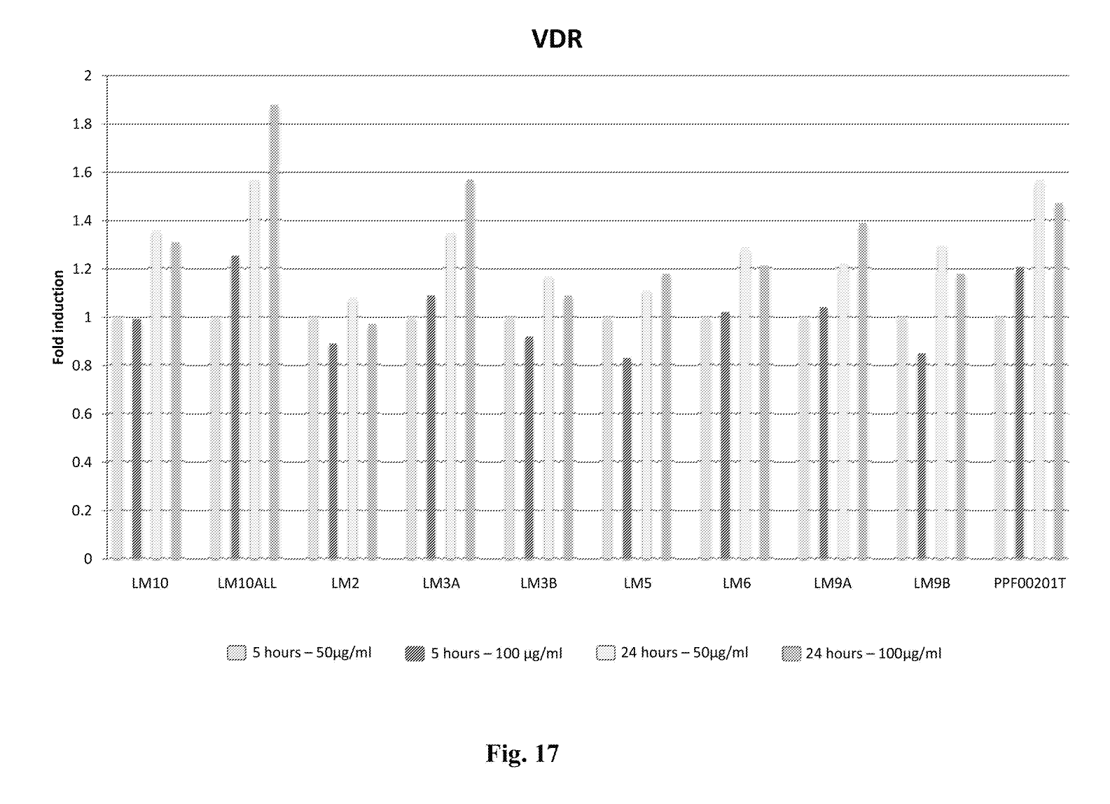

The effects of the compositions of the present teachings on VDR gene expression are shown in FIG. 17. The VDR (vitamin D receptor) is required for β-catenin induced hair follicle formation in adult epidermis. VDR is a TCF/Lef-independent transcriptionnal effector of the Wnt pathway. VDR is a TCF/Lef independent transcriptional effector of the canonical Wnt pathway that promotes HF differentiation

VDR expression was not significantly modified after short duration treatment (4 h) with all tested fractions. For long duration treatment (24 h), fractions LM10, LM3A, PPF 002-01 T and particularly LM10ALL induced a marked increase in VDR expression with a slight dose-dependency for fractions LM10ALL and LM3A.

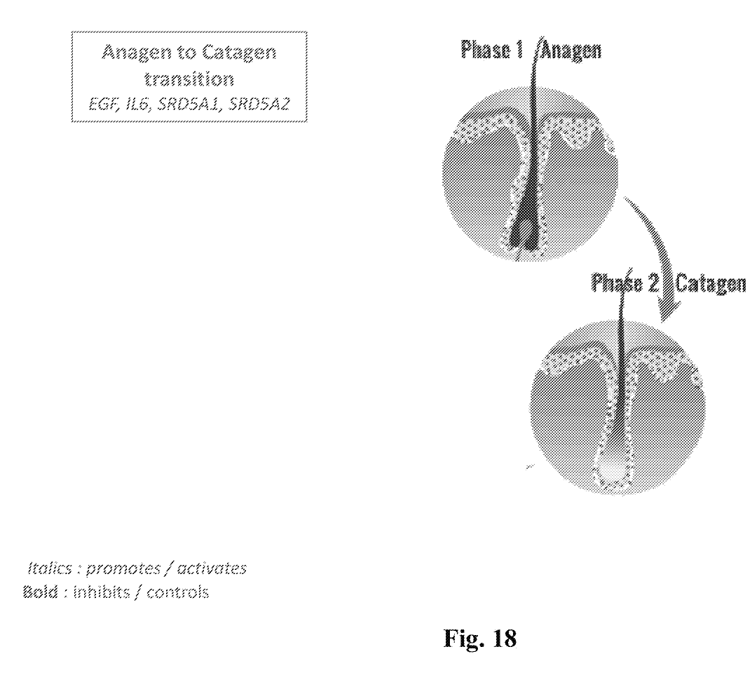

Genes that are relevant to anagen to catagen transition are shown in FIG. 18, and the effects of the compositions of the present teachings on these genes are summarized in Table 3.

TABLE 3
Summary of attenuating activity of the mulateiro extract of the present teachings
on the expression of genes relevant to anagen to catagen transition.

| | | |
|---|---|---|
| Gene | Activity | Role |

Anagen to catagen transition

| | | |
|---|---|---|
| EGF | + | EGF was shown to retard hair growth; the EGF receptor mediates |
| | | the termination of the anagen stage. |
| IL6 | + | IL-6 expression induces anagen to catagen progression. The |
| | | presence of DHT causes upregulation of IL-6 which inhibits the |
| | | hair shaft elongation. |
| SRD5A1 and | + | 5 alpha reductases 1 and 2 both catalyzes the conversion of |
| SRD5A2 | | testosterone into the more potent androgen, dihydrotestosterone |
| | | (DHT). DHT causes miniaturization of the hair follicles and hair loss |
| | | by shortening the anagen of the hair cycle, causing miniaturization |
| | | of the follicles, and producing progressively shorter, finer hairs (in |
| | | link with IL-6 upregulation). |

In Table 3 the presence of extract induced attenuating activity is indicated with a (+)-sign, the absence—with a (−)-sign.

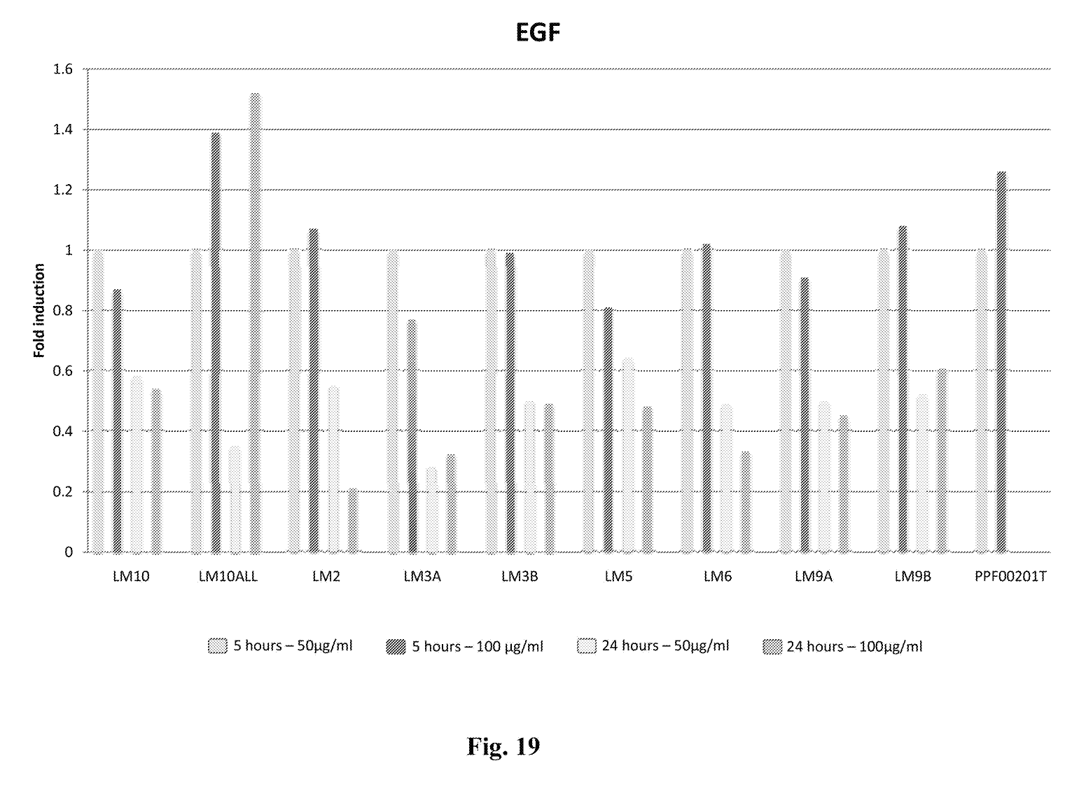

The effects of the compositions of the present teachings on EGF gene expression are shown in FIG. 19. EGF molecules promote robust keratinocyte proliferation. Epithelial growth factor (EGF) was shown to retard hair growth. The EGF receptor mediates the termination of the anagen stage. EGF is central in the regulation of hair morphogenesis, with its cyclical on/off switch being important for the progression of the hair cycle. On the other hand, continuous expression of EGF, or TGF-α, although producing a wavy phenotype, impedes the growth of hair. Therefore, cyclic variations in the level of EGFR, which is a key intermediate in signal transmission, may result in hair growth and produce new hair formation.

While most fractions inducef a decrease in EGF expression especially for long duration treatment, LM10ALL at 100 μm/ml (whatever the duration of treatment) induced an increase 1.5 fold-induction) in EGF expression. The inhibition in EGF expression is particularly marked for LM2 (80% decrease at 100 μm/ml—24 h treatment).

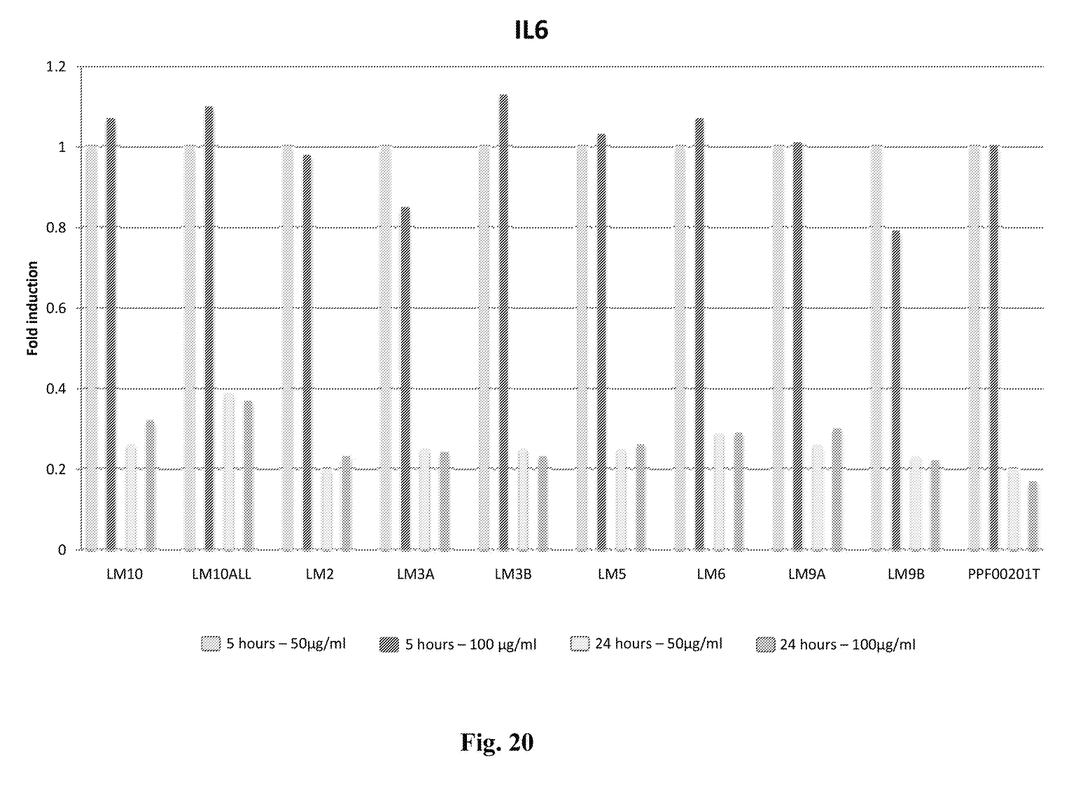

The effects of the compositions of the present teachings on IL6 gene expression are shown in FIG. 20. IL-6 expression induces anagen to catagen progression. The presence of DHT causes upregulation of IL-6 that in turn promotes the expression of IL-6 receptor along with gp130 in keratinocytes and matrix cells. The outcome of this IL-6 upregulated expression is inhibition of the hair shaft elongation with simultaneous expression of matrix cell proliferation.

Long term treatment with both tested doses induced a marked decrease in IL-6 expression for all fractions tested.

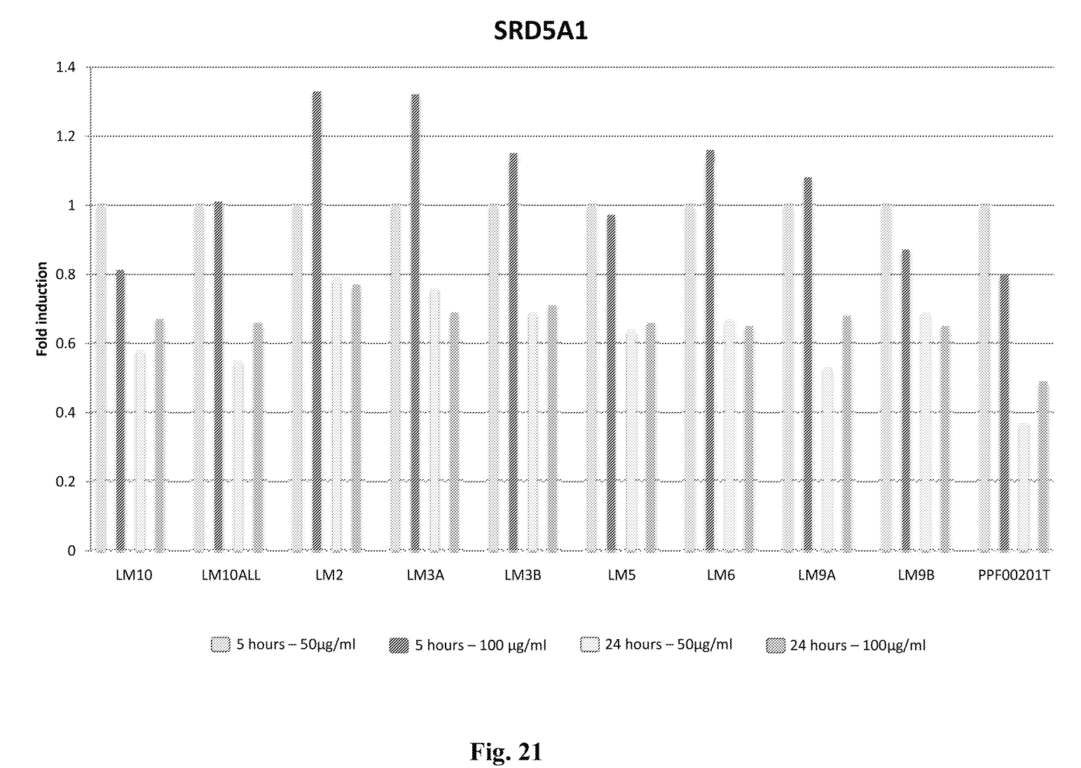

The effects of the compositions of the present teachings on SRD5A1 gene expression are shown in FIG. 21. 5 alpha reductases 1 and 2 both catalyze the conversion of testosterone into the more potent androgen, dihydrotestosterone (DHT). DHT causes miniaturization of the hair follicles. DHT causes hair loss by shortening the growth, or anagen, phase of the hair cycle, causing miniaturization (decreased size) of the follicles, and producing progressively shorter, finer hairs. Eventually these hairs totally disappear.

While some fractions induce an increase in SRD5A1 (5 alpha reductase 1) expression after short duration treatment at 100 μg/ml (mostly LM2 & LM3A), all fractions promote a significant decrease in its expression after 24 h for both tested doses. This decrease is particularly marked for PPF 002-01 T that induce a 50% to 60% inhibition;

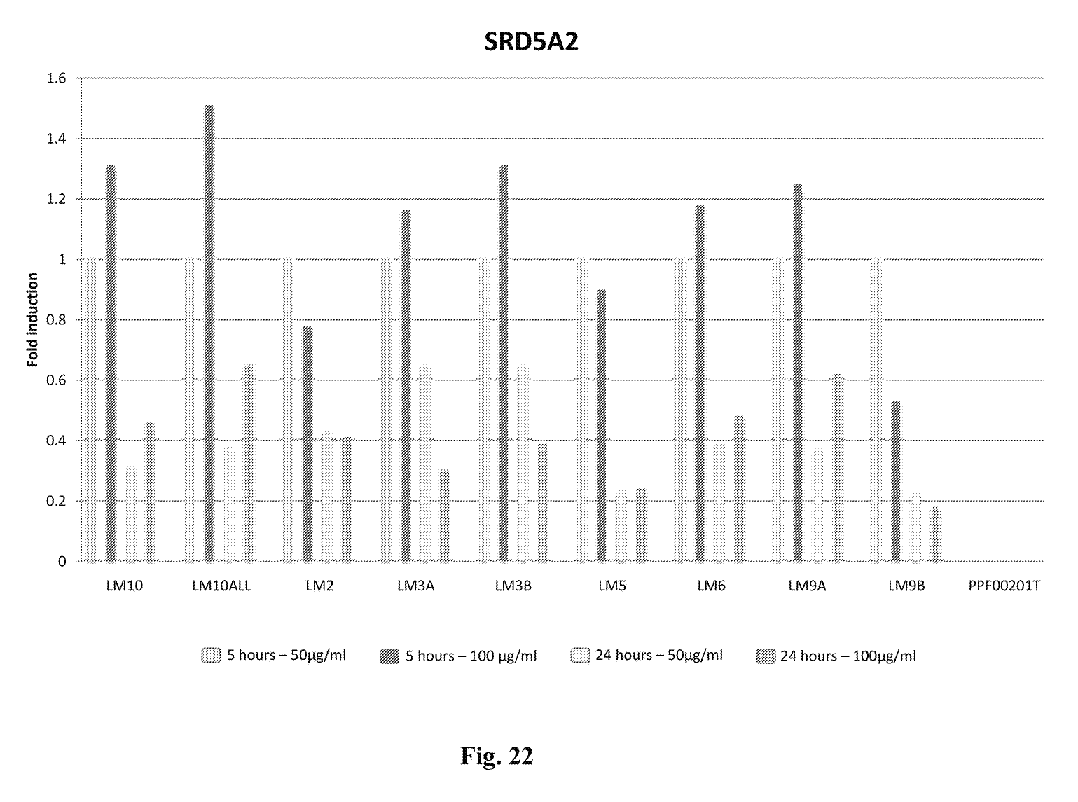

The effects of the compositions of the present teachings on SRD5A2 (5 alpha reductase 2) gene expression are shown in FIG. 22. Short duration treatment with the different fractions tested induces variable effects ranging form slight inhibition to moderate activation (LM10ALL) of SRD5A2 expression. Conversely, after 24 h of treatment, all fractions promote a significant decrease in SRD5A2 expression. This effect is particularly marked for fractions LM5 and LM9B that induce a nearly 80% inhibition in SRD5A2 expression.

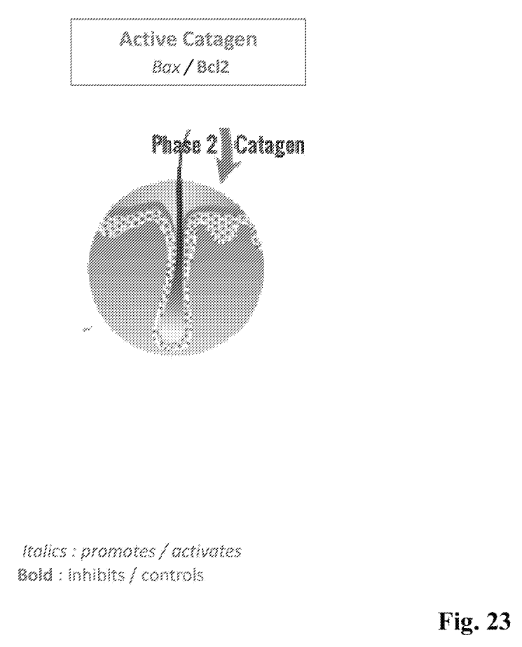

Genes that are relevant to active catagen phase are shown in FIG. 23, and the effects of the compositions of the present teachings on these genes are summarized in Table 4.

TABLE 4
Summary of attenuating activity of the mulateiro extract of the present
teachings on the expression of genes relevant to active catagen phase.

| | | |
|---|---|---|
| Gene | Activity | Role |

Active Catagen

| | | |
|---|---|---|
| Bax | + | Catagen is an apoptosis-driven process accompanied by terminal |
| | | differentiation, proteolysis, and matrix remodeling. In male |
| | | pattern baldness, testosterone delivered to hair follicles is |
| | | converted to DHT by type II 5a-reductase; DHT then stimulates the |
| | | synthesis of TGF-β2 in dermal papilla cells; TGF-β2 induces |
| Bcl-2 | − | epithelial cells to promote up-regulation and activation of caspase- |
| | | 9 and caspase-3 in matrix cells, resulting in the removal of |
| | | epithelial cells by apoptotic cell death. |
| | | Bax (pro-apoptotic) and Bcl-2 (anti-apoptotic) are regulators of the |
| | | intrinsic apoptotic pathway. |

In Table 4 the presence of extract induced attenuating activity is indicated with a (+)-sign, the absence—with a (−)-sign.

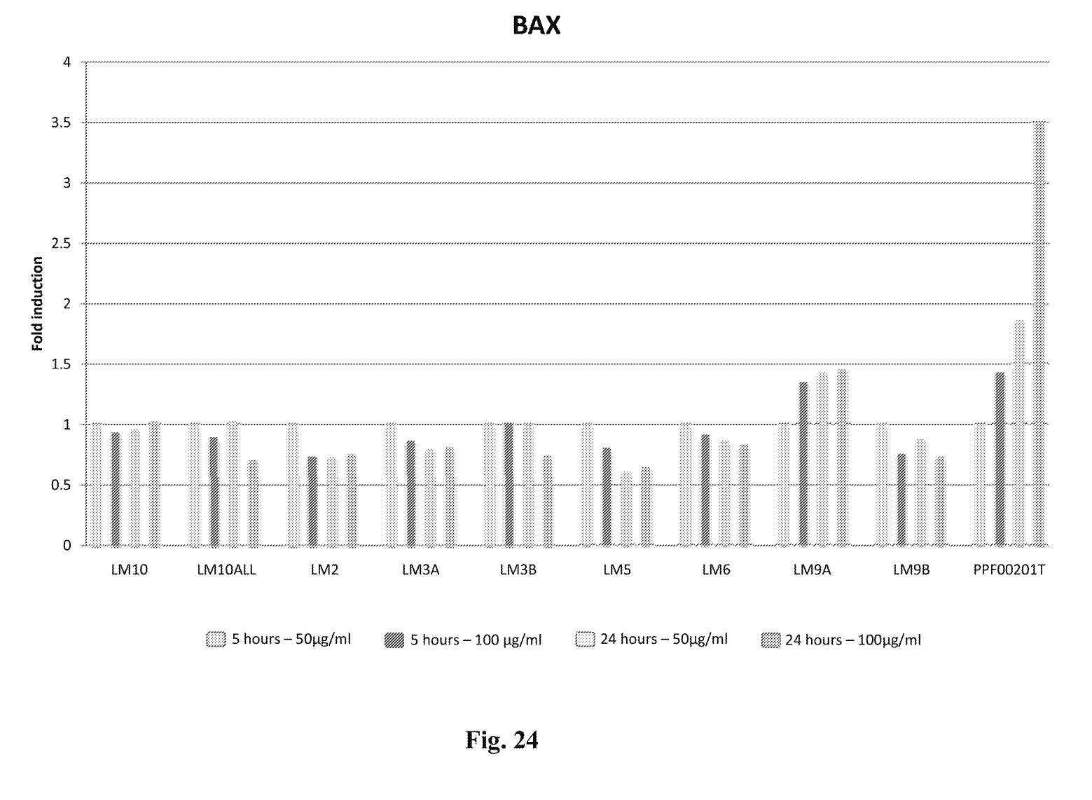

The effects of the compositions of the present teachings on BAX gene expression are shown in FIG. 24. Bax/Bcl-2 are regulators of the intrinsic apoptotic pathway. In catagen, epithelial components are eliminated through a typical apoptotic process. In males pattern baldness, testosterone delivered to hair follicles is converted to DHT by type II 5a-reductase; DHT then stimulates the synthesis of TGF-β2 in dermal papilla cells; TGF-β2 induces epithelial cells to promote up-regulation and activation of caspase-9 and caspase-3 in matrix cells, resulting in the removal of epithelial cells by apoptotic cell death. Bax is a promoter of the apoptotic intrinsic pathway

Only LM9A and PPF00201T exhibit an increase in Bax expression for all conditions tested; a dose and time dependent increase is particularly marked for PPF00201T. For all other fractions tested, a slight to moderate decrease in Bax expression is observed (LM5 showing the highest decrease near 50% for long duration treatment).

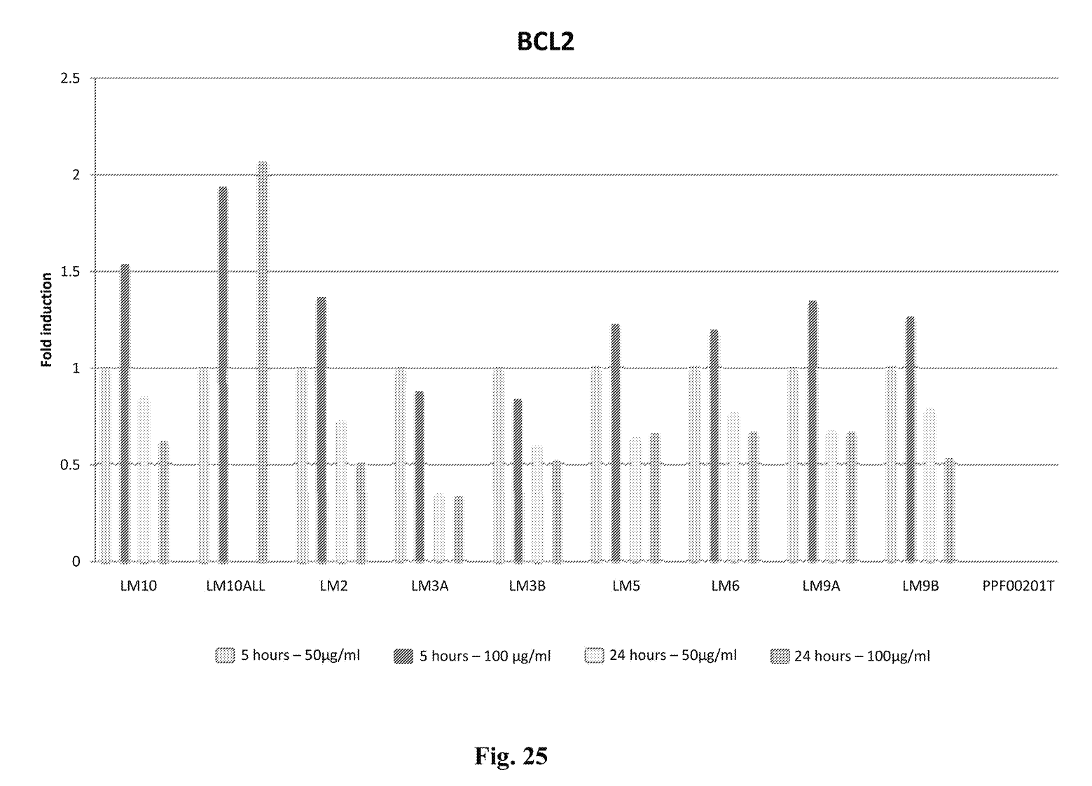

The effects of the compositions of the present teachings on Bcl2 gene expression are shown in FIG. 25. Bcl-2 is an inhibitor of the intrinsic apoptotic pathway.

For most tested fractions (except LM3A and LM3B), a short duration treatment at 100 μg/ml induced a moderate increase in Bcl2 expression, whereas long duration treatment (24 h) systematically induced a marked decrease for both tested concentrations, except for fraction LM10ALL that exhibited a significant increase in Bcl2 expression for both short and long duration treatments at 100 μg/ml.

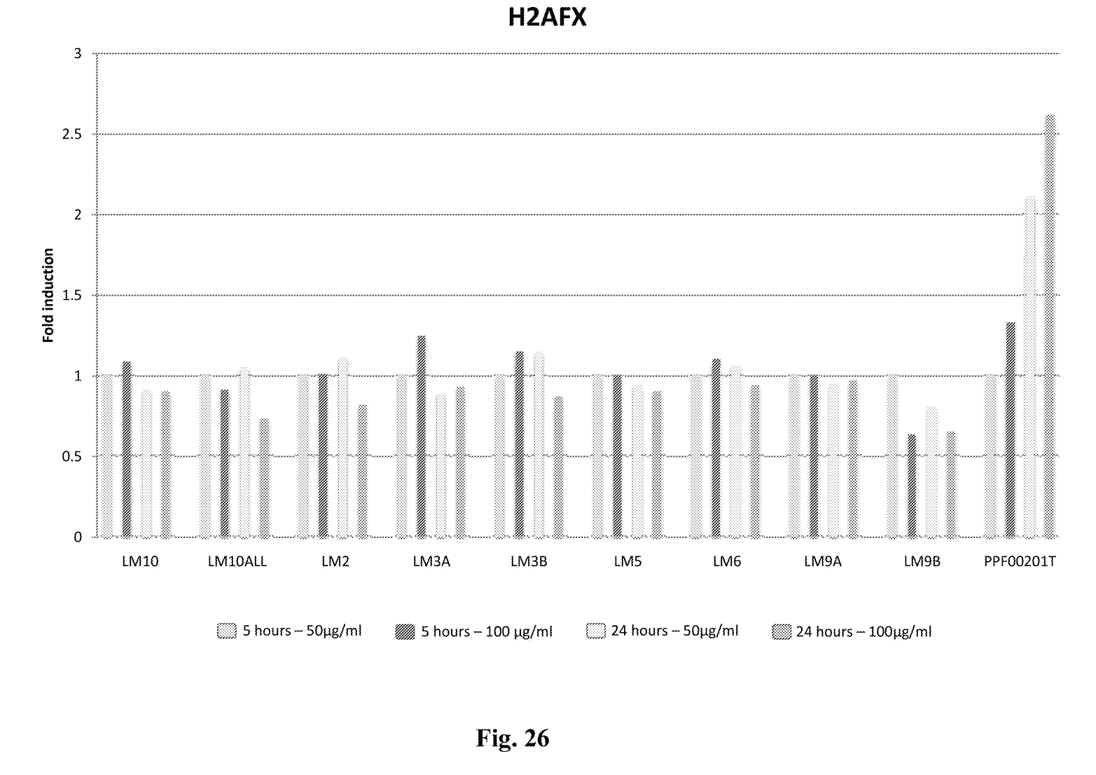

The effects of the compositions of the present teachings on H2AFX gene expression are shown in FIG. 26. Nuclear phosphorylated γH2AX is indicative of DNA double-strand breaks and is a marker of senescence. Senescence marker are observed in progressive hair loss

No appreciable variation, except for PPF00201T, especially for long term treatment 24 h (both tested doses), that induces a significant increase in H2AFX expression.

### EXAMPLES

The following Examples illustrate the forgoing aspects and other aspects of the present teachings. These non-limiting Examples are put forth so as to provide those of ordinary skill in the art with illustrative embodiments as to how the compounds, compositions, articles, devices, and/or methods claimed herein are made and evaluated. The Examples are intended to be purely exemplary of the teachings and are not intended to limit the scope of what the inventors regard as their invention. Efforts have been made to ensure accuracy with respect to numbers (e.g., amounts, temperature, etc.) but some errors and deviations should be accounted for.

### Example 1: Preparation of the Mulateiro Bark Extract (Batch PPF002-01T) and its Fractions

The preparation procedure for the Mulateiro bark extract (batch No. PPF002-01T) is illustrated in FIG. 1. The extract was further resolved by liquid chromatography into fractions containing major components. The fractions were designated LM10, LM10ALL, LM2, LM3A, LM3B, LM5, LM6, LM9A, and LM9B. The bark extract and the fractions were used in subsequent activity tests.

### Example 2: Test System Preparation—Co-Culture of Human Fibroblasts and Keratinocytes

Human Fibroblasts:

Human Fibroblasts: Dermal human fibroblasts obtained from outgrowth of explant of foreskin and cultured in DMEM/Ham's F12, 1:1, v/v and a 15 mmol/1 HEPES buffer system, supplemented with 50U/ml penicillin, 0.05 mg/ml streptomycin and FCS (10% v/v).

Human Keratinocytes:

Skin grafts were obtained from patients undergoing plastic surgery breast reductions and abdominoplasties (all patients gave informed consent for the use of tissues for research that were not needed for clinical diagnosis) or foreskin. Samples of this skin were cut into 0.5 cm² pieces using a scalpel blade and were incubated overnight (18 h) at 4° C. in 10 ml 0.15% w/v trypsin. FCS was added to neutralize the trypsin and the epidermal and dermal layers were carefully separated using a pair of forceps with fine points. A scalpel blade was used to gently scrape basal keratinocytes from the undersurface of the epidermis and the papillary surface of the dermis. The cells were collected into universal containers in a 1:1 mixture of FCS and PBS. The cell suspension was then centrifuged at 200 g for 5 min and cells were resuspended in either a known volume in culture medium is MCDB 153 supplemented with EGF (5 ng/mL), Insulin (5 μg/mL), Hydrocortisone (5 ng/mL), BPE (70 μg/mL) (bovine pituitary extract).

Co-Culture of Fibroblasts (Passage 2) and Keratinocytes (Passage 2)

50×10³ cells per well were seeded as individual cultures and also as 1:1 co-cultures in various culture media on 6-well plates for keratinocyte culture and is; MCDB-153 medium, which was developed for in vitro keratinocytes culture, and DMEM/Ham's F12, 1:1 (v/v) and a 15 mmol/1 HEPES buffer system, supplemented with 50U/ml penicillin, 0.05 mg/ml streptomycin and FCS (10% v/v). Cultures are incubated, at 37° C. in a humidified atmosphere containing 5% (v/v).

Cell Culture Treatment

Two treatment periods were evaluated: 5 hours and 24 hours in duration. Four series of testing were carried out: three series of the test substance (10, 50 and 100 μg/ml) in duplicates; a first negative control (n=3); and a second negative control (n=3). These four series were incubated for 5 hours and 24 h at 37° C. in a humid atmosphere containing 5% (v/v) CO2. Thereafter cells were treated with TRIzol®.

### Example 3: Quantitative Real-Time PCR (q-PCR)

Gene expression levels of the genes of interest were evaluated utilizing q-PCR techniques, essentially as described below. Cell samples were homogenized in Tri-reagent (Euromedex, France) and RNA was isolated using a standard chloroform/isopropanol protocol (Chomczynski P, Sacchi N. Single-step method of RNA isolation by acid guanidinium thiocyanate-phenol-chloroform extraction. Anal Biochem. 1987 April; 162(1):156-9). RNA was processed and analyzed following an adaptation of published methods (Bustin S A, Benes V, Garson J A, Hellemans J, Huggett J, Kubista M, Mueller R, Nolan T, Pfaffl M W, Shipley G L, Vandesompele J, Wittwer C T. The MIQE guidelines: minimum information for publication of quantitative real-time PCR experiments. Clin Chem. 2009 April; 55(4):611-22.). cDNA was synthesized from 2 μg of total RNA using RevertAid Premium Reverse Transcriptase (Fermentas) and primed with oligo-dT primers (Fermentas) and random primers (Fermentas). Q-PCR was performed using a LightCycler® 480 Real-Time PCR System (Roche, Meylan, France). QPCR reactions were done in duplicate for each sample, using transcript-specific primers, cDNA (4 ng) and LightCycler 480 SYBR Green I Master (Roche) in a final volume of 10 μl. The PCR data were exported and analyzed in an informatics tool (Gene Expression Analysis Software Environment) developed at the NeuroCentre Magendie (Bordeaux, France). For the determination of the reference gene, the Genorm method was used. Relative expression analysis was corrected for PCR efficiency and normalized against two reference genes. The ribosomal protein L13a (RPL13A) and succinate dehydrogenase complex, subunit A (SDHA) genes were used as reference genes. The relative level of expression was calculated using the comparative (2-ΔΔCT) method (Livak K J, Schmittgen T D. Analysis of relative gene expression data using real-time quantitative PCR and the 2(-Delta Delta C(T)) Method. Methods. 2001 December; 25(4):402-8.). Primer sequences used are listed in Table 5 below.

TABLE 5
Primers used for q-PCT testing.

| Gene | GenBank ID | Forward Sequence (5′-3′) | Reverse Sequence (5′-3′) |
|---|---|---|---|
| SDHA | NM_004168 | CTGTCTTCATACGCTTCTGCACTC | CCAGCCACTAGGTGCCAATC |
| RPL13A | NM_012423 | GGGAGCAAGGAAAGGGTCTTA | CACCTGCACAATTCTCCGAGT |
| GSK3B | NM_002093 | CCAGTCATCTTGTCTGCACCAA | GAAATGCCAGTGTCTTCATATCCA |
| CTNNB1 | NM_001904 | CAGCTGCTGTTTTGTTCCGAA | CAGCTCAACTGAAAGCCGTTT |
| WNT10A | NM_025216 | GTCTGTGATCGCCGGACAGT | GTCAGTCCTAGAGCCCACAGAAG |
| KRT15 | NM_002275 | CCACTCTCATCAGGCCAAGTG | TGAAGGCAGGGACTGGAGTT |
| PDGFA | NM_002607 | CCCGCAGTGCACACCTAGA | ACACAGACAGAAGCGGCAATG |
| PDGFC | NM_016205 | ACGGCTTAGGGTAATGTCAGTACAG | TAAGCAAGGCAACGGAATCAG |
| BMP2 | NM_001200 | CCCAACACGCAGCAAAATTA | GCAAGCTGATAGGTCAGAGAACAG |
| EGF | NM_001963 | ACAGGAGGCTTCGGAGTTTCT | GCAATCACACCAAGAGGGAAAA |
| LEF1 | NM_016269 | AGGACGGTAACTTGGCTGCAT | GTGCTGATGGATGTGCTGGTT |
| TCF3 | NM_003200 | CCCCAGACCAAACTGCTCAT | CTCGCACTTGCTGCTCCAA |
| TCF4 | NM_001083962 | AACACTGAAGCTATGCATTTGAAGA | GACTTAGCAACCTGCAGCACAA |
| Srd5a1 | NM_001047 | TGCTGTGTGTAAGTGGAGAACTTG | GCCTTTGCCTCACCTTGGA |
| SRD5A2 | NM_000348 | CCACATTTCCACACCAGAACTG | GGTGACCCCTTCACAAGAGTTT |
| FGF-7 | NM_002009 | GGCCTCCATCCCTCTTACTCA | CAGCTGCGTGACCTTAGGTGTA |
| IL6 | NM_000600 | GCATGGGCACCTCAGATTGT | TGCCCAGTGGACAGGTTTCT |
| VDR | NM_000376 | GGCAGGAATGTGTGGCAGAT | AGCAGGCACTGTTTACATCCTTT |
| H2afx | NM_002105 | GCACTTGGTAACAGGCACATCTT | CTCTGCCCTCCCCTAAATGTC |
| Bcl2 | NM_000633 | CAAGCAAACATCCTATCAACAACAA | TTCCATCCTCCACCAGTGTTC |

1-13. (canceled)

14. A pharmaceutical composition for hair loss prevention, containing a pharmaceutically effective amount of Mulateiro bark extract or one or more Mulaterio bark extract components, the components selected from the group consisting of: chlorogenic acid (5-caffeoyl quinic acid) represented by formula 1: 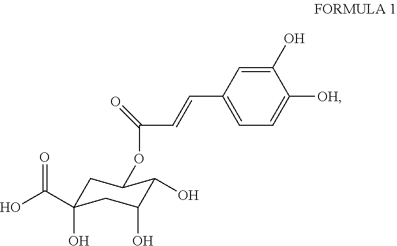 [SMILES: O=C(/C=C/c1ccc(O)c(O)c1)OC1CC(O)(C(=O)O)CC(O)C1O]
neochlorogenic acid (3-caffeoyl quinic acid) represented by formula 2: 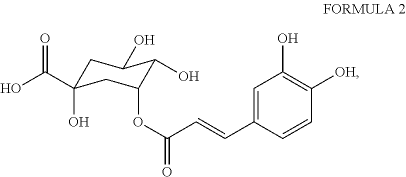 [SMILES: O=C(/C=C/c1ccc(O)c(O)c1)OC1CC(O)(C(=O)O)CC(O)C1O]
beta-morriniside represented by formula 3: 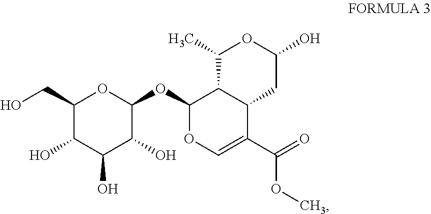 [SMILES: COC(=O)C1=CO[C@@H](O[C@@H]2O[C@H](CO)[C@@H](O)[C@H](O)[C@H]2O)[C@@H]2[C@H](C)O[C@H](O)C[C@H]12]
kingiside represented by formula 4: 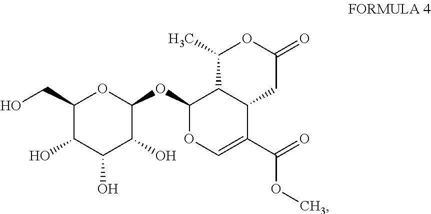
loganin (loganitin glucoside) represented by formula 5: 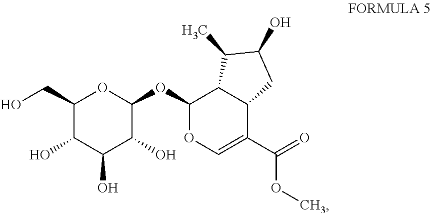 [SMILES: COC(=O)C1=CO[C@@H](O[C@@H]2O[C@H](CO)[C@@H](O)[C@H](O)[C@H]2O)[C@@H]2[C@@H](C)[C@@H](O)C[C@H]12]
diderroside represented by formula 6: 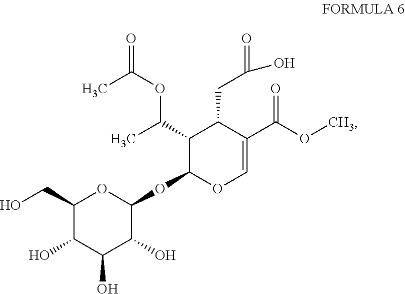 [SMILES: COC(=O)C1=CO[C@@H](O[C@@H]2O[C@H](CO)[C@@H](O)[C@H](O)[C@H]2O)[C@H](C(C)OC(C)=O)[C@@H]1CC(=O)O]
secoxyloganin represented by formula 7: 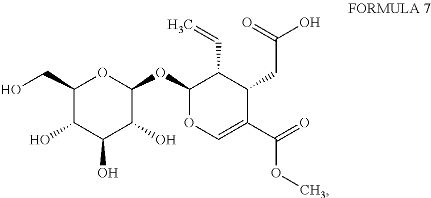 [SMILES: C=C[C@H]1[C@H](O[C@@H]2O[C@H](CO)[C@@H](O)[C@H](O)[C@H]2O)OC=C(C(=O)OC)[C@H]1CC(=O)O]
containing 6-tigloyl diderroside represented by formula 8: 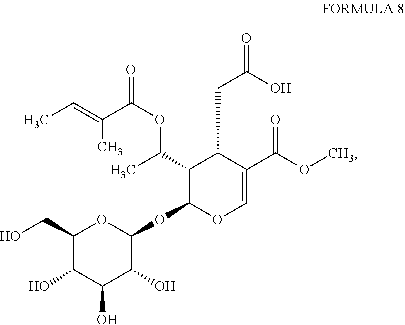 [SMILES: C/C=C(\C)C(=O)OC(C)[C@H]1[C@H](O[C@@H]2O[C@H](CO)[C@@H](O)[C@H](O)[C@H]2O)OC=C(C(=O)OC)[C@H]1CC(=O)O]
cryptochlorogenic acid (4-caffeoyl quinic acid) represented by formula 9: 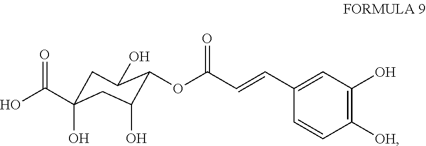 [SMILES: O=C(/C=C/c1ccc(O)c(O)c1)OC1C(O)CC(O)(C(=O)O)CC1O]
and combinations thereof; and a pharmaceutically acceptable carrier.

15. A pharmaceutical composition for promotion of hair growth, containing a pharmaceutically effective amount of Mulateiro bark extract or one or more Mulaterio bark extract components, the components selected from the group consisting of: chlorogenic acid (5-caffeoyl quinic acid) represented by formula 1: 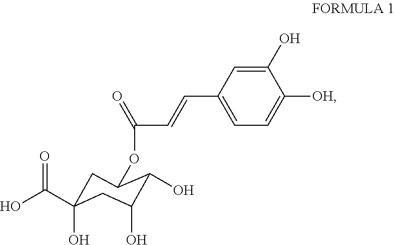 [SMILES: O=C(/C=C/c1ccc(O)c(O)c1)OC1CC(O)(C(=O)O)CC(O)C1O]
neochlorogenic acid (3-caffeoyl quinic acid) represented by formula 2: 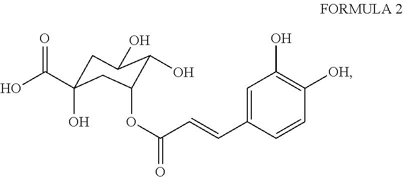 [SMILES: O=C(/C=C/c1ccc(O)c(O)c1)OC1CC(O)(C(=O)O)CC(O)C1O]
beta-morriniside represented by formula 3: 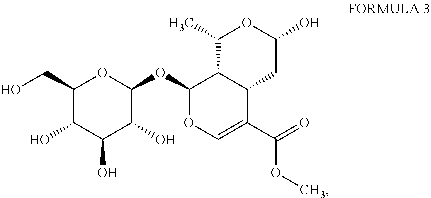 [SMILES: COC(=O)C1=CO[C@@H](O[C@@H]2O[C@H](CO)[C@@H](O)[C@H](O)[C@H]2O)[C@@H]2[C@H](C)O[C@H](O)C[C@H]12]
kingiside represented by formula 4: 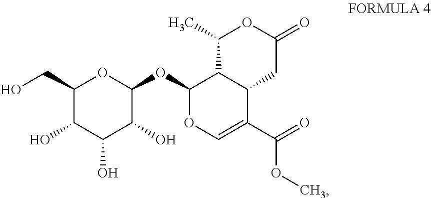
loganin (loganitin glucoside) represented by formula 5: 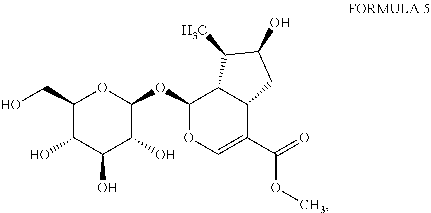 [SMILES: COC(=O)C1=CO[C@@H](O[C@@H]2O[C@H](CO)[C@@H](O)[C@H](O)[C@H]2O)[C@@H]2[C@@H](C)[C@@H](O)C[C@H]12]
diderroside represented by formula 6: 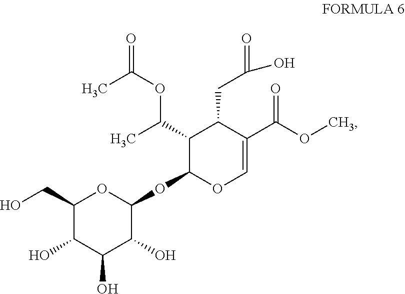 [SMILES: COC(=O)C1=CO[C@@H](O[C@@H]2O[C@H](CO)[C@@H](O)[C@H](O)[C@H]2O)[C@H](C(C)OC(C)=O)[C@@H]1CC(=O)O]
secoxyloganin represented by formula 7: 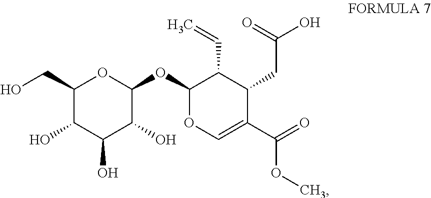 [SMILES: C=C[C@H]1[C@H](O[C@@H]2O[C@H](CO)[C@@H](O)[C@H](O)[C@H]2O)OC=C(C(=O)OC)[C@H]1CC(=O)O]
containing 6-tigloyl diderroside represented by formula 8: 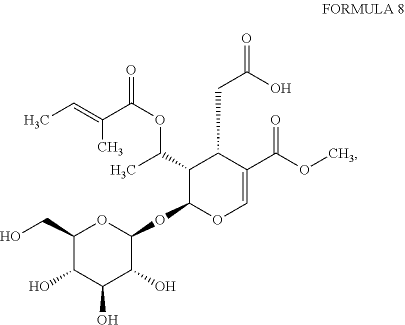 [SMILES: C/C=C(\C)C(=O)OC(C)[C@H]1[C@H](O[C@@H]2O[C@H](CO)[C@@H](O)[C@H](O)[C@H]2O)OC=C(C(=O)OC)[C@H]1CC(=O)O]
cryptochlorogenic acid (4-caffeoyl quinic acid) represented by formula 9: 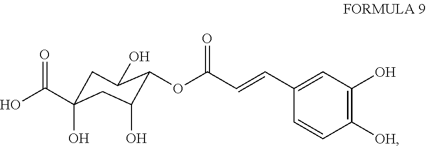 [SMILES: O=C(/C=C/c1ccc(O)c(O)c1)OC1C(O)CC(O)(C(=O)O)CC1O]
and combinations thereof; and a pharmaceutically acceptable carrier.

16. A method for preventing hair loss, including administering a therapeutically effective dose to a patient in need thereof of a pharmaceutical composition containing a Mulateiro bark extract.

17. A method for promoting hair growth, including administering a therapeutically effective dose to a patient in need thereof of a pharmaceutical composition containing a Mulateiro bark extract.
